SEQUENCE LISTING (33 sequences)

SEQ ID NO: 1 - RNA, 25 nt, synthetic construct
  source 1..25: mol_type = other RNA
cctcctcgcctggttctggaagaca

SEQ ID NO: 2 - RNA, 20 nt, synthetic construct
  source 1..20: mol_type = other RNA
ttggcccctcctcgcctggt

SEQ ID NO: 3 - RNA, 20 nt, synthetic construct
  source 1..20: mol_type = other RNA
ctgcagttggcccctcctcg

SEQ ID NO: 4 - RNA, 20 nt, synthetic construct
  source 1..20: mol_type = other RNA
tcaaacccctcacctgcagt

SEQ ID NO: 5 - RNA, 25 nt, synthetic construct
  source 1..25: mol_type = other RNA
cctgcagttggcccctcctcgcctg

SEQ ID NO: 6 - RNA, 25 nt, synthetic construct
  source 1..25: mol_type = other RNA
cccctcacctgcagttggcccctcc

SEQ ID NO: 7 - RNA, 17 nt, synthetic construct
  source 1..17: mol_type = other RNA
gcagttggcccctcctc

SEQ ID NO: 8 - RNA, 25 nt, synthetic construct
  source 1..25: mol_type = other RNA
cacctgcagttggcccctcctcgcc

SEQ ID NO: 9 - RNA, 25 nt, synthetic construct
  source 1..25: mol_type = other RNA
atcctcccacagagcctgtacggag

SEQ ID NO: 10 - RNA, 25 nt, synthetic construct
  source 1..25: mol_type = other RNA
ccctgatcctcccacagagcctgta

SEQ ID NO: 11 - RNA, 25 nt, synthetic construct
  source 1..25: mol_type = other RNA
ggcgttgccgcctttgccacaagat

SEQ ID NO: 12 - RNA, 25 nt, synthetic construct
  source 1..25: mol_type = other RNA
ctcacctctcctctcctcggcgttg

SEQ ID NO: 13 - RNA, 25 nt, synthetic construct
  source 1..25: mol_type = other RNA
ctccactcacctctcctctcctcgg

SEQ ID NO: 14 - RNA, 20 nt, synthetic construct
  source 1..20: mol_type = other RNA
agcagggcggctgtccccag

SEQ ID NO: 15 - RNA, 25 nt, synthetic construct
  source 1..25: mol_type = other RNA
gccacaagatgaccccaatgagcag

SEQ ID NO: 16 - RNA, 25 nt, synthetic construct
  source 1..25: mol_type = other RNA
cctttgccacaagatgaccccaatg

SEQ ID NO: 17 - RNA, 25 nt, synthetic construct
  source 1..25: mol_type = other RNA
tgccgcctttgccacaagatgaccc

SEQ ID NO: 18 - RNA, 25 nt, synthetic construct
  source 1..25: mol_type = other RNA
tcctcggcgttgccgcctttgccac

SEQ ID NO: 19 - RNA, 13 nt, synthetic construct
  source 1..13: mol_type = other RNA
tttgccacaagat

SEQ ID NO: 20 - RNA, 20 nt, synthetic construct
  source 1..20: mol_type = other RNA
ccgcctttgccacaagatga

SEQ ID NO: 21 - RNA, 25 nt, synthetic construct
  source 1..25: mol_type = other RNA
tttcttgttgaccatccccccagtc

SEQ ID NO: 22 - RNA, 25 nt, synthetic construct
  source 1..25: mol_type = other RNA
ccatccccccagtcacatgtgttgg

SEQ ID NO: 23 - RNA, 25 nt, synthetic construct
  source 1..25: mol_type = other RNA
cctcctcgcctggttctggaagaca

SEQ ID NO: 24 - RNA, 20 nt, synthetic construct
  source 1..20: mol_type = other RNA
ctggcccctcatcgccggtt

SEQ ID NO: 25 - RNA, 20 nt, synthetic construct
  source 1..20: mol_type = other RNA
cttcagctggcccctcatcg

SEQ ID NO: 26 - RNA, 20 nt, synthetic construct
  source 1..20: mol_type = other RNA
tccagtccctcaccttcagc

SEQ ID NO: 27 - RNA, 25 nt, synthetic construct
  source 1..25: mol_type = other RNA
tgggttgtcgttttcgccacaggat

SEQ ID NO: 28 - RNA, 23 nt, synthetic construct
  source 1..23: mol_type = other RNA
actactcccaggcctcccaggat

SEQ ID NO: 29 - RNA, 22 nt, synthetic construct
  source 1..22: mol_type = other RNA
gcagggctactactcccaggca

SEQ ID NO: 30 - RNA, 25 nt, synthetic construct
  source 1..25: mol_type = other RNA
tctcactcacctctcctcacgcctg

SEQ ID NO: 31 - RNA, 25 nt, synthetic construct
  source 1..25: mol_type = other RNA
cccagactcacccacagagcctgta

SEQ ID NO: 32 - RNA, 127 nt, Homo sapiens
  source 1..127: mol_type = mRNA
gctctgtgggaggatcagggctgggaactctagccctggccctggggatcctgggaggcctggggacagccgccctgctcattggggtcatcttgtggcaaaggcggcaacgccgaggagaggagag

SEQ ID NO: 33 - RNA, 18 nt, synthetic construct
  source 1..18: mol_type = other RNA
tcgttttcgccacaggat

(19) United States
(12) Patent Application Publication
(10) Pub. No.: US 2026/0308597 A1
(43) Pub. Date: Oct. 8, 2026

(54) METHODS OF MINIMISING EFFECTS OF SMOKE EXPOSURE

(71) Applicant: RAGE BIOTECH PTY LTD, Melbourne, Victoria (AU)

(72) Inventors: Merlin Christopher Thomas, Melbourne (AU); Raelene Jane Pickering, Melbourne (AU)

(73) Assignee: RAGE BIOTECH PTY LTD, Melbourne, Victoria (AU)

(21) Appl. No.: 19/481,491

(22) PCT Filed: May 17, 2024
(86) PCT No.: PCT/AU2024/050501

§ 371 Date: Nov. 4, 2025

Related U.S. Application Data
(63) Continuation-in-part of application No. PCT/AU2023/050962, filed on Oct. 5, 2023.

(30) Foreign Application Priority Data
May 19, 2023 (AU) 2023901559

(51) Int. Cl. C12N 15/113; A61P 11/00

(52) U.S. Cl. CPC C12N 15/1138; A61P 11/00; C12N 2310/11; C12N 2310/315; C12N 2310/3181; C12N 2310/321; C12N 2310/3231; C12N 2310/3233; C12N 2310/3513; C12N 2310/3519; C12N 2320/32; C12N 2320/33

(57) ABSTRACT

The present invention relates to compositions, methods and kits for the treatment or prevention of effects of smoke exposure. In particular, the compositions, methods and kits are particularly useful, but not limited to, the treatment or prevention of acute or chronic smoke exposure. In one aspect, the present invention provides a method of treating or preventing obstructive pulmonary disease, associated with or caused by exposure to smoke, in a subject in need thereof, the method comprising administering to the subject an antisense oligonucleotide (AON) that promotes the production of endogenous soluble RAGE, treating or preventing obstructive pulmonary disease, associated with or caused by exposure to smoke in the subject.

### FIELD OF THE INVENTION

The present invention relates to compositions, methods and kits for the treatment or prevention of effects of smoke exposure. In particular, the compositions, methods and kits are particularly useful, but not limited to, the treatment or prevention of acute or chronic smoke exposure.

### RELATED APPLICATIONS

This application claims the benefit of priority from Australian provisional application no. 2023901559 filed 19 May 2023, and International application no. PCT/AU2023/050962 filed 5 Oct. 2023, the entire disclosures of which are incorporated herein by reference.

### BACKGROUND OF THE INVENTION

Smoke exposure leads to inflammation in the airway and causes the migration of neutrophils and monocytes, amongst other proinflammatory cells, into the lung which release cytotoxic enzymes and other oxidative agents. The accumulation of these toxic substances is a major contributor to the damage to the airway epithelium and endothelium of the lung parenchyma. As a consequence, gas exchange by the injured alveoli is impaired, leading to a range of obstructive pulmonary diseases.

The most common obstructive pulmonary disease associated with smoke exposure is chronic obstructive pulmonary disease (COPD) which affects about 30 million people in the US and hundreds of millions worldwide. The most common cause of COPD is cigarette smoking, and the disease often progresses even after smoking cessation. COPD encompasses two lung pathologies that together make up this disease, including chronic bronchitis and emphysema. Chronic bronchitis involves the large and small airways of the lungs, which undergo thickening of the epithelium, increased mucus production, and increased airway rigidity. Emphysema involves the loss of elasticity in the alveoli, that results in “dead space” in the lungs, preventing exhalation of full breaths of fresh air. Many patients have a some combination of these two pathologies. COPD is medically managed using bronchodilators, beta-agonists, and anti-inflammatory agents, as well as oxygen in advanced cases. These treatments do not significantly impact the long term decline in lung function characteristic of this disease and there is no cure.

There is a need for new or improved methods for the treatment and/or prevention of obstructive pulmonary disease, associated with or caused by exposure to smoke.

Reference to any prior art in the specification is not an acknowledgment or suggestion that this prior art forms part of the common general knowledge in any jurisdiction or that this prior art could reasonably be expected to be understood, regarded as relevant, and/or combined with other pieces of prior art by a skilled person in the art.

### SUMMARY OF THE INVENTION

In one aspect, the present invention provides a method of treating or preventing obstructive pulmonary disease, associated with or caused by exposure to smoke, in a subject in need thereof, the method comprising administering to the subject an antisense oligonucleotide (AON) that promotes the production of endogenous soluble RAGE and/or reduces the production of membrane bound RAGE (mRAGE), thereby treating or preventing obstructive pulmonary disease, associated with or caused by exposure to smoke in the subject.

In another aspect, the invention also provides a method of alleviating or ameliorating a symptom of obstructive pulmonary disease, associated with or caused by exposure to smoke, in a subject in need thereof, the method comprising administering to the subject in need thereof an antisense oligonucleotide (AON) that promotes the production of endogenous soluble RAGE and/or reduces the production of membrane bound RAGE (mRAGE), thereby alleviating or ameliorating a symptom of obstructive pulmonary disease, associated with or caused by exposure to smoke in the subject.

In another aspect, the invention also provides use of an antisense oligonucleotide (AON) that promotes the production of endogenous soluble RAGE and/or reduces the production of membrane bound RAGE (mRAGE) in the manufacture of a medicament for the treatment or prevention of obstructive pulmonary disease, associated with or caused by exposure to smoke, in a subject in need thereof.

In another aspect, the invention also provide use of an antisense oligonucleotide (AON) that promotes the production of endogenous soluble RAGE and/or reduces the production of membrane bound RAGE (mRAGE) in the manufacture of a medicament for alleviating or ameliorating a symptom of obstructive pulmonary disease, associated with or caused by exposure to smoke, in a subject in need thereof.

In another aspect, the invention also provide an antisense oligonucleotide (AON) that promotes the production of endogenous soluble RAGE and/or reduces the production of membrane bound RAGE (mRAGE) for use in a method of treating or preventing obstructive pulmonary disease, associated with or caused by exposure to smoke, in a subject in need thereof, the method comprising administering to the subject in need thereof an antisense oligonucleotide (AON), thereby treating or preventing obstructive pulmonary disease, associated with or caused by exposure to smoke in the subject.

In another aspect, the invention also provide an antisense oligonucleotide (AON) that promotes the production of endogenous soluble RAGE and/or reduces the production of membrane bound RAGE (mRAGE) for use in a method for alleviating or ameliorating a symptom of obstructive pulmonary disease, associated with or caused by exposure to smoke, in a subject in need thereof, the method comprising administering to the subject in need thereof an antisense oligonucleotide (AON), thereby alleviating or ameliorating a symptom of obstructive pulmonary disease, associated with or caused by exposure to smoke in the subject.

In another aspect, the present invention provides a method for the treatment or prevention of obstructive pulmonary disease, associated with or caused by exposure to smoke, in a subject in need thereof, the method comprising the steps of
identifying a subject having obstructive pulmonary disease, associated with or caused by exposure to smoke; and
administering to the subject in need thereof an antisense oligonucleotide (AON) that promotes the production of endogenous soluble RAGE and/or reduces the production of membrane bound RAGE (mRAGE),
   thereby treating or preventing obstructive pulmonary disease, associated with or caused by exposure to smoke in the subject.

In another aspect, the present invention provides an antisense oligonucleotide (AON) that promotes the production of endogenous soluble RAGE and/or reduces the production of membrane bound RAGE (mRAGE) for use in a method for the treatment or prevention of obstructive pulmonary disease, associated with or caused by exposure to smoke, in a subject in need thereof, the method comprising the steps of
identifying a subject having obstructive pulmonary disease, associated with or caused by exposure to smoke; and
administering to the subject in need thereof the antisense oligonucleotide (AON),
   thereby treating or preventing obstructive pulmonary disease, associated with or caused by exposure to smoke in the subject.

In one aspect, the present invention provides a method of treating or preventing respiratory inflammation associated with or caused by exposure to smoke in a subject in need thereof, the method comprising administering to the subject an antisense oligonucleotide (AON) that promotes the production of endogenous soluble RAGE and/or reduces the production of membrane bound RAGE (mRAGE), thereby treating or preventing respiratory inflammation associated with or caused by exposure to smoke in the subject.

In another aspect, the invention also provides a method of alleviating or ameliorating a symptom of respiratory inflammation associated with or caused by exposure to smoke, in a subject in need thereof, the method comprising administering to the subject in need thereof an antisense oligonucleotide (AON) that promotes the production of endogenous soluble RAGE and/or reduces the production of membrane bound RAGE (mRAGE), thereby alleviating or ameliorating a symptom of respiratory inflammation associated with or caused by exposure to smoke in a subject in need thereof.

In another aspect, the invention also provides use of an antisense oligonucleotide (AON) that promotes the production of endogenous soluble RAGE and/or reduces the production of membrane bound RAGE (mRAGE) in the manufacture of a medicament for the treatment or prevention of respiratory inflammation associated with or caused by exposure to smoke in a subject in need thereof.

In another aspect, the invention also provides use of an antisense oligonucleotide (AON) that promotes the production of endogenous soluble RAGE and/or reduces the production of membrane bound RAGE (mRAGE) in the manufacture of a medicament for alleviating or ameliorating a symptom of respiratory inflammation associated with or caused by exposure to smoke, in a subject in need thereof.

In another aspect, the invention also provides an antisense oligonucleotide (AON) that promotes the production of endogenous soluble RAGE and/or reduces the production of membrane bound RAGE (mRAGE) for use in a method for the treatment or prevention of respiratory inflammation associated with or caused by exposure to smoke in a subject in need thereof, the method comprising administering to the subject in need thereof the antisense oligonucleotide, thereby treating or preventing respiratory inflammation associated with or caused by exposure to smoke in the subject.

In another aspect, the invention also provides an antisense oligonucleotide (AON) that promotes the production of endogenous soluble RAGE and/or reduces the production of membrane bound RAGE (mRAGE) for use in a method for alleviating or ameliorating a symptom of respiratory inflammation associated with or caused by exposure to smoke, in a subject in need thereof, the method comprising administering to the subject in need thereof the antisense oligonucleotide, thereby alleviating or ameliorating a symptom of respiratory inflammation associated with or caused by exposure to smoke in a subject in need thereof.

In another aspect, the present invention provides a method for the treatment or prevention of respiratory inflammation associated with or caused by exposure to smoke in a subject in need thereof, the method comprising the steps of
identifying a subject having respiratory inflammation associated with or caused by exposure to smoke; and
administering to the subject in need thereof an antisense oligonucleotide (AON) that promotes the production of endogenous soluble RAGE and/or reduces the production of membrane bound RAGE (mRAGE),
   thereby treating or preventing respiratory inflammation associated with or caused by exposure to smoke in the subject.

In another aspect, the present invention provides an antisense oligonucleotide (AON) that promotes the production of endogenous soluble RAGE and/or reduces the production of membrane bound RAGE (mRAGE) for use in a method for the treatment or prevention of respiratory inflammation associated with or caused by exposure to smoke in a subject in need thereof, the method comprising the steps of
identifying a subject having respiratory inflammation associated with or caused by exposure to smoke; and
administering to the subject in need thereof the antisense oligonucleotide (AON),
   thereby treating or preventing respiratory inflammation associated with or caused by exposure to smoke in the subject.

In any aspect, the subject has, or is at risk of, an obstructive lung disorder, or respiratory inflammation, associated with or caused by exposure to smoke and has not substantially reduced their exposure to smoke.

In any aspect, the subject has, or is at risk of, an obstructive lung disorder, or respiratory inflammation, associated with or caused by exposure to smoke and is currently being exposed to smoke. Preferably, the smoke exposure is of level that if left untreated causes, or enhances, the progression of the obstructive lung disorder or respiratory inflammation (e.g. if a method or use of the invention is not applied).

In any embodiment, the subject is currently being exposed to cigarette smoke or tobacco smoke, or vaping. Preferably, the subject is currently a cigarette smoker or tobacco smoker, or vaper, (i.e. an active smoker or vaper) such as a chronic (long-term) cigarette smoker or tobacco smoker, or vaper.

In any aspect, the subject has, or is at risk of, an obstructive lung disorder, or respiratory inflammation, associated with or caused by exposure to smoke and will be further exposed to smoke. Preferably, the smoke exposure is of level that if left untreated causes, or enhances, the progression of the obstructive lung disorder or respiratory inflammation (e.g. if a method or use of the invention is not applied).

In any aspect, the AON that promotes the production of endogenous soluble RAGE is administered directly to the airway and/or lungs.

For any aspect of the invention, the administration to the airway and the lungs is via any route that allows an antisense oligonucleotide (AON) that promotes the production of endogenous soluble RAGE and/or reduces the production of membrane bound RAGE (mRAGE) to contact the airway, lung or a part thereof. For example, an antisense oligonucleotide (AON) that promotes the production of endogenous soluble RAGE and/or reduces the production of membrane bound RAGE (mRAGE) may be administered via any route such that there is improvement in a symptom of the disease or condition to be treated or prevented. Preferably, the route of administration allows exposure to the respiratory tract or lung parenchyma such as alveolar tissue with respiratory bronchioles, alveolar ducts and terminal bronchioles. Preferably, an antisense oligonucleotide (AON) that promotes the production of endogenous soluble RAGE is formulated for inhalation or intranasal administration.

In any aspect or embodiment, the antisense oligonucleotide (AON) that promotes the production of endogenous soluble RAGE and/or reduces the production of membrane bound RAGE (mRAGE) is administered via inhalation or intranasal administration.

In another aspect, the invention provides a kit or article of manufacture comprising any an antisense oligonucleotide (AON) that promotes the production of endogenous soluble RAGE and/or reduces the production of membrane bound RAGE (mRAGE), and/or a pharmaceutical composition comprising an antisense oligonucleotide (AON) that promotes the production of endogenous soluble RAGE and/or reduces the production of membrane bound RAGE (mRAGE).

In any aspect or embodiment of the invention, the obstructive pulmonary disease is chronic obstructive pulmonary disease (COPD), emphysema, combined pulmonary fibrosis and emphysema (CPFE), bronchitis, respiratory bronchiolitis/respiratory bronchiolitis-interstitial lung disease, bronchiectasis, asthma, lung, tracheal cancer or a smoking-related interstitial lung diseases (SR-ILDs).

In any embodiment of the invention, the chronic obstructive smoking-related interstitial lung diseases (SR-ILDs) is selected from pulmonary Langerhans cell histiocytosis, desquamative interstitial pneumonitis, and acute eosinophilic pneumonia.

In any aspect, a method or use of the invention may promote the production of endogenous soluble RAGE and/or reduces the production of membrane bound RAGE (mRAGE) in a subject, particularly the respiratory tract of the subject. In any embodiment, a method of the invention may promote the production of endogenous soluble RAGE in one or more tissues of the respiratory tract, for example one or more of the tissues of the respiratory tract described herein.

In any aspect, an AON may promote the production of endogenous soluble RAGE by promoting the inclusion of exon 9b and/or the exclusion (e.g. skipping) of exon 10. Therefore, in any aspect, the AON may promote splicing in the RAGE pre-mRNA resulting in the inclusion of exon 9b and/or skipping of exon 10. For example, the AON may result in an increase of the level of RAGE_v1, RAGE_v6, RAGE_v8, RAGE_v9 RAGE_v10, RAGE_v15, RAGE_v18, and RAGE_v19 mRNA, preferably an increase in the level of RAGE_v1, RAGE_v6, RAGE_v8, RAGE_v9, RAGE_v10, RAGE_v15, RAGE_v18, and RAGE_v19 mRNA in one or more tissues of the respiratory tract.

In any aspect, the AON is administered to the total respiratory tract, the upper respiratory tract or the lower respiratory tract.

As used herein, the upper respiratory tract may include any one or more of the following regions: the nose and nasal passages, paranasal sinuses, the pharynx, and the portion of the larynx above the vocal folds (cords). Typically, the lower respiratory tract includes any one or more of the following regions: the portion of the larynx below the vocal folds, trachea, bronchi and bronchioles. The lungs can be included in the lower respiratory tract and include the respiratory bronchioles, alveolar ducts, alveolar sacs, and alveoli.

In any aspect, the AON is administered as an aerosol, a dry powder or as nasal drops. In any embodiment, the AON may be administered using a nasal spray pump, intranasal installation, intratrachael instillation, metered dose inhaler (MDI), dry powder inhaler (DPI), nebuliser (jet, ultrasonic mesh or vibrating mesh) or soft mist inhaler (SMI).

In any aspect, the AON is an AON of 10 to 50 nucleotides comprising a targeting sequence complementary to a region near or within an intron of the RAGE pre-mRNA. Alternatively, the AON is 10 to 50 nucleotides comprising a targeting sequence complementary or adjacent to a splice site of the RAGE pre-mRNA.

Because factors such as RNA secondary structure, competition between AONs and SR proteins, heterogeneous nuclear ribonucleoproteins (hnRNPs), and/or other elements that make up the spliceosome can affect AONs' action, AONs directed at the crucial acceptor or donor splice sites will not always alter splicing. Consequently, in any aspect of the invention, the AON is 10 to 50 nucleotides comprising a targeting sequence complementary or adjacent to cis-acting RNA elements in the pre-mRNA of RAGE that act as enhancers or silencers, that, when bound by an elements of the splicosome (e.g. protein-splicing factors, uRNA, lncRNA) modulates the splicing of a nearby exon.

In any aspect, the AON is 10 to 50 nucleotides comprising a targeting sequence complementary to RAGE pre-mRNA which modulates secondary structure of said mRNA to influence splice site selection.

In any aspect, the AON is an isolated or purified AON for inducing exclusion (also known as skipping) of one or more exonic sequences in the RAGE gene transcript or part thereof.

In any aspect, the AON is an isolated or purified AON for inducing retention of intronic sequences in the RAGE gene transcript or part thereof.

In any aspect, the AON comprises at least one modified nucleotide. Typically, the AON is chemically-modified to prevent degradation of the pre-mRNA-AON complex, including but not limited to phosphorodiamidate morpholino oligomers (PMO), 2′ O-methyl phosphorothioate oligonucleotides (20Me PS), and 2′-O-methoxyethyl phosphorothioate oligonucleotides (2′-MOE PS), locked nucleic acid (LNA) modified AONs, thermostable twisted intercalating nucleic acid (TINA) and peptide nucleic acids (PNAs).

In any aspect, the AON comprises at least one modified nucleotide selected from the group consisting of: phosphorodiamidate morpholino oligomers (PMO), 2′ O-methyl oligonucleotides (20Me), 2′-O-methoxyethyl oligonucleotides (2′-MOE), phosphorothioate oligonucleotides, locked nucleic acid (LNA) modified AONs, thermostable twisted intercalating nucleic acid (TINA) and peptide nucleic acids (PNAs).

In any aspect, the AONs may be conjugated to moieties to increase their delivery, including but not limited to cell-penetrating peptides (CPPs), vivo-morpholinos (VMO) or peptide phosphorodiamidate morpholino oligomers (PPMO).

In any aspect, the antisense oligonucleotide comprises, consists essentially of or consists of a nucleotide sequence at least 80%, 81%, 82%, 83%, 84%, 85%, 86%, 87%, 88%, 89%, 90%, 91%, 92%, 93%, 94%, 95%, 96%, 97%, 98%, 99% or 100% complementary to a target region of exon 10 of RAGE pre-mRNA over the entirety of the antisense oligonucleotide. Preferably, the 5′-most nucleotide of the AON is nucleotide position 88 or 114 of exon 10 or is between nucleotide positions 88 to 114 of exon 10. Preferably, the 5′-most nucleotide of the target region is nucleotide position 88 or 114 of exon 10 or is between nucleotide positions 88 to 114 of exon 10.

In any embodiment, the 5′-most nucleotide of the target region is position 88 or 113, or between nucleotide positions 88 to 113 of exon 10 of RAGE pre-mRNA.

In any embodiment, the 5′-most nucleotide of the target region is position 90 or 113, or between nucleotide positions 90 to 113 of exon 10 of RAGE pre-mRNA.

In any embodiment, the 5′-most nucleotide of the target region is position 88 or 108, or between nucleotide positions 88 to 108 of exon 10 of RAGE pre-mRNA.

In any embodiment, the 5′-most nucleotide of the target region is position 90 or 108, or between nucleotide positions 90 to 108 of exon 10 of RAGE pre-mRNA.

In any embodiment, the 5′-most nucleotide of the target region is position 88 or 95, or between nucleotide positions 88 to 95 of exon 10 of RAGE pre-mRNA.

In any embodiment, the target region is from nucleotide position 88 to 137 of exon 10 of RAGE pre-mRNA.

In any embodiment, the target region is from nucleotide position 88 to 107 of exon 10 of RAGE pre-mRNA.

In any embodiment, the target region is from nucleotide position 90 to 102 of exon 10 of RAGE pre-mRNA.

In any embodiment, the target region is from nucleotide position 95 to 119 of exon 10 of RAGE pre-mRNA.

In any embodiment, the target region is between nucleotide positions 108 to 132 of exon 10 of RAGE pre-mRNA.

In any embodiment, the target region is between nucleotide positions 113 to 137 of exon 10 of RAGE pre-mRNA.

In any aspect, the AON may be 8 to 40 nucleotides in length, 15 to 25 nucleotides in length or 18 nucleotides in length.

In any embodiment, the AON is selected from the group comprising the sequences set forth in any of Tables 1a-1d. Preferably, the AON is selected from the list comprising: SEQ ID NO: 1-31 and 33, for example, the AON is SEQ ID NO: 11, 18, 19, or 20 or a nucleotide sequence at least 85%, 90% or 95% identical thereto.

In any embodiment, the AON may be selected to be an AON capable of binding to a selected target site, wherein the target site is a putative mRNA splicing site selected from a splice donor site, splice acceptor site, splice enhancer sequences splice silencer sequences or sites that modulate the secondary structure of pre-mRNA. The target site may also include some flanking intronic sequences when the donor or acceptor splice sites are targeted.

More specifically, the AON may be selected from the group comprising of any one or more of SEQ ID NOs: 1-31 and 33 and/or the sequences set forth in any of Tables 1a-1d, and combinations or cocktails thereof. More preferably, the AON is SEQ ID NO: 11, 18, 19, or 20. The combination of AONs is preferably a combination of SEQ ID NO: 11 and 10, or SEQ ID NO: 11 and 13. This includes sequences which can hybridise to such sequences under stringent hybridisation conditions, sequences complementary thereto, sequences containing modified bases, modified backbones, and functional truncations or extensions thereof which possess or modulate pre-mRNA processing activity in a RAGE gene transcript.

In certain embodiments, AONs may be 100% complementary to the target sequence, or may include mismatches, e.g. to accommodate variants, as long as a hetero-duplex formed between the oligonucleotide and target sequence is sufficiently stable to withstand the action of cellular nucleases and other modes of degradation which may occur in vivo. Hence, certain oligonucleotides may have about or at least about 70% sequence complementarity, e.g. 70%, 71%, 72%, 73%, 74%, 75%, 76%, 92%, 93%, 94%, 95%, 96%, 97%, 98%, 99% or 100% sequence complementarity, between the oligonucleotide and the target sequence.

The methods and uses of the invention extends also to a combination of two or more AONs capable of binding to a selected target to modulate alternative splicing of the RAGE pre-mRNA, including a construct comprising two or more such AONs. The constructs may be used together for a combined AON-based therapy. The combination of AONs is preferably a combination of SEQ ID NO: 11 and 10, or SEQ ID NO: 11 and 13.

As used herein, except where the context requires otherwise, the term “comprise” and variations of the term, such as “comprising”, “comprises” and “comprised”, are not intended to exclude further additives, components, integers or steps.

Further aspects of the present invention and further embodiments of the aspects described in the preceding paragraphs will become apparent from the following description, given by way of example and with reference to the accompanying drawings.

### BRIEF DESCRIPTION OF THE DRAWINGS

FIG. 1. Experimental timeline used in the smoke-induced inflammation mouse model studies.

FIG. 2. Mouse weights. Percentage change in relation to do. Ave±95% CI, n=8/group. Statistical significance for % weight change between groups was determined by one-way ANOVA without correction for multiple comparisons.

FIG. 3. Preliminary BALF cell counts. Group 1 is represented by red datapoints, and Group 2 is represented by blue datapoints. Data is mean±95% CI, n=8/group. Statistical significance for % weight change between groups was determined by one-way ANOVA without correction for multiple comparisons. *p<0.05, **p<0.01, ***p<0.001, ****p<0.0001.

FIG. 4. Acute CSE causes acute lung inflammation. Mice were exposed to Air or CSE (9 cigarettes/day, 4 days). Differential counts in the bronchoalveolar lavage fluid (BALF). Group 1 is represented by red datapoints, and Group 2 is represented by blue datapoints. Data is mean±95% CI, n=8/group. Statistical significance for % weight change between groups was determined by two-way ANOVA without correction for multiple comparisons. *p<0.05, **p<0.01, ***p<0.001.

FIG. 5. RAGE_v1 mRNA expression in the left lung after a single dose of ASO 6713 or saline vehicle. C57BL/6 mice were intratracheally administered ASO 6713 (3 mg/kg), ASO 6713 (10 mg/kg) or saline and RAGE_v1 mRNA levels (encoding esRAGE) was measured in the samples collected from the left lung at 11 days, 14 days, or 17 days following administration. Data shows mean±SEM; *p<0.05 vs control.

FIG. 6. Treatment with AON-m79 prevents airways inflammation, lung injury and experimental COPD in mice exposed to cigarette smoke for 8-weeks. (A) Cell counts in bronchoalveolar lavage fluid, (B) airway epithelial thickness, (C) small airway sub-epithelial collagen deposition, (D) alveolar diameter and (E) lung function in terms of total lung capacity. Data show mean±SEM; *p<0.05 vs control; #p<0.05 vs smoke group.

FIG. 7. RAGE exon 10 mRNA expression in the left lung after a single dose of ASO 6713 or saline vehicle. C57BL/6 mice were intratracheally administered ASO 6713 (3 mg/kg), ASO 6713 (10 mg/kg) or saline and RAGE exon 10 mRNA levels (encoding membrane bound RAGE, mRAGE) was measured in the samples collected from the left lung at 11 days, 14 days, or 17 days following administration. Data shows mean±SEM; *p<0.05 vs control.

FIG. 8. Experimental timeline used in the extended smoke-induced inflammation mouse model studies.

FIG. 9. Treatment with ASO 6713 attenuates emphysema-like alveolar enlargement. The mean linear intercept is a morphometric measure of airspace enlargement that is widely used for the quantification of injury in lung histopathology images. An increase in the mean linear intercept identifies animals with increased lung damage. C57BL/6 mice were exposed to air or CSE (9 cigarettes/day) for 4 or 8 months. Mice were separated into the treatment groups of 1) Negative control—Air+saline, 2) Positive control—CSE+saline, 3) CSE+ASO 6713, 4) CSE+ASO delayed, 5) Smoking cessation+ASO 6713 and 6) Smoking cessation+saline. Mice were administered ASO 6713 (3 mg/kg) or saline according to the protocol for each treatment group as outlined in Example 5 below. The mean linear intercept was measured. Smoking cessation after 4 months (groups 5 and 6) was also associated with lesser lung damage when compared to 8-months of cigarette smoke exposure. Data shows mean±SEM; *p<0.05 vs control; **p<0.01 vs control; ***p<0.001 vs control; ****p<0.0001 vs control.

### DETAILED DESCRIPTION OF THE EMBODIMENTS

It will be understood that the invention disclosed and defined in this specification extends to all alternative combinations of two or more of the individual features mentioned or evident from the text or drawings. All of these different combinations constitute various alternative aspects of the invention.

Reference will now be made in detail to certain embodiments of the invention. While the invention will be described in conjunction with the embodiments, it will be understood that the intention is not to limit the invention to those embodiments. On the contrary, the invention is intended to cover all alternatives, modifications, and equivalents, which may be included within the scope of the present invention as defined by the claims.

One skilled in the art will recognize many methods and materials similar or equivalent to those described herein, which could be used in the practice of the present invention. The present invention is in no way limited to the methods and materials described. It will be understood that the invention disclosed and defined in this specification extends to all alternative combinations of two or more of the individual features mentioned or evident from the text or drawings. All of these different combinations constitute various alternative aspects of the invention.

All of the patents and publications referred to herein are incorporated by reference in their entirety.

For purposes of interpreting this specification, terms used in the singular will also include the plural and vice versa.

The present invention is based on the surprising finding that administration of an antisense oligonucleotide (AON) that that promotes the production of endogenous soluble RAGE and/or reduces the production of membrane bound RAGE (mRAGE) can prevent or treat smoke induced inflammation. In particular, administration of an AON that promotes the production of endogenous soluble RAGE and/or reduces the production of membrane bound RAGE (mRAGE) to the respiratory tract in a preclinical model of acute smoke exposure was able to inhibit inflammation.

### Indications

The present invention relates to methods of treating or preventing obstructive pulmonary (lung) diseases associated with or caused by exposure to smoke. Preferably the exposure is an acute or chronic exposure to smoke.

Such obstructive disease may result from narrowing of the smaller bronchi and larger bronchioles of the lung and are generally characterised by inflamed and easily collapsible airways, obstruction to airflow, problems exhaling and frequent medical clinical visits and hospitalisations.

Examples of obstructive pulmonary diseases which can be treated or prevented in accordance with the instant methods are chronic obstructive pulmonary disease (COPD) including cigarette smoke-induced COPD, emphysema, combined pulmonary fibrosis and emphysema (CPFE), bronchitis, respiratory bronchiolitis/respiratory bronchiolitis-interstitial lung disease, bronchiectasis, asthma, lung, tracheal cancer or a smoking-related interstitial lung diseases (SR-ILDs). In any embodiment of the invention, the chronic obstructive smoking-related interstitial lung diseases (SR-ILDs) is pulmonary Langerhans cell histiocytosis, desquamative interstitial pneumonitis, or acute eosinophilic pneumonia.

The skilled person will be familiar with the various classifications of obstructive pulmonary disease. COPD is also known as chronic obstructive airways disease (COAD) or chronic airflow limitation (CAL). The skilled person will appreciate that in common usage, the term COPD encompasses emphysema and chronic bronchitis. Chronic bronchitis is typically diagnosed where a patient suffers from chronic cough, mucus production, or both, for at least three months in at least two successive years where other causes of chronic cough have been excluded. In chronic bronchitis, airway obstruction is caused by chronic and excessive secretion of abnormal airway mucus, inflammation, and bronchospasm. Often chronic bronchitis is exacerbated by frequent or chronic infection.

Emphysema involves the destruction of elastin in terminal bronchioles, which leads to remodelling, destruction and ultimate collapse of the airway walls. Patients with emphysema gradually lose the ability to exhale, causing a rise in blood waste gasses (such as carbon dioxide), a drop in blood oxygen, and a general degradation of patient stamina and overall health. A characteristic of emphysema is permanent loss of alveoli. Remodelling leads to permanent enlargement of the air spaces distal to the terminal bronchioles, and destruction of terminal bronchiole walls, though without fibrosis. Emphysema is progressive with a poor prognosis. Since there is no known method for repairing elastin or restoring the alveoli, therapy is generally palliative and persistent. Most patients suffering from COPD have both emphysema and chronic bronchitis.

Bronchiectasis refers to the abnormal, irreversible dilatation of the bronchi caused by destructive and inflammatory changes in the airway walls. Bronchiectasis has three major anatomical patterns: cylindrical bronchiectasis, varicose bronchiectasis and cystic bronchiectasis.

Chronic bronchitis is defined as the presence of chronic cough and sputum production for at least three months of two consecutive years in the absence of other diseases recognized to cause sputum production. In chronic bronchitis, epidemiologically the bronchial epithelium becomes chronically inflamed with hypertrophy of the mucus glands and an increased number of goblet cells. The cilia are also destroyed and the efficiency of the mucociliary escalator is greatly impaired. Mucus viscosity and mucus production are increased, leading to difficulty in expectorating. Pooling of the mucus leads to increased susceptibility to infection. Microscopically there is infiltration of the airway walls with inflammatory cells. Inflammation is followed by scarring and remodeling that thickens the walls and also results in narrowing of the airways. As chronic bronchitis progresses, there is squamous metaplasia (an abnormal change in the tissue lining the inside of the airway) and fibrosis (further thickening and scarring of the airway wall). The consequence of these changes is a limitation of airflow. Repeated infections and inflammation over time leads to irreversible structural damage to the walls of the airways and to scarring, with narrowing and distortion of the smaller peripheral airways.

Asthma is defined as a chronic inflammatory condition of the airways, leading to widespread and variable airways obstruction that is reversible spontaneously or with treatment. In some patients with chronic asthma, the disease progresses, leading to irreversible airway obstruction, particularly if the asthma is untreated, either because it has not been diagnosed or mismanaged, or if it is particularly severe. The airway inflammation in asthma over time can lead to remodeling of the airways through increased smooth muscle, disruption of the surface epithelium increased collagen deposition and thickening of the basement membrane.

Symptoms of the above mentioned obstructive airway diseases include airway obstruction, inflammation, coughing, wheezing, hyperplasia and hypersecretion of mucous glands, excessive mucous production, productive cough, haemoptysis (coughing up blood or blood-stained mucus from the bronchi, larynx, trachea, or lungs) and dyspnea (shortness of breath).

As used herein, an obstructive pulmonary disease “associated” with exposure to smoke includes any obstructive pulmonary disease which results from, is caused by, or suspected of being caused by exposure to smoke.

As used herein, “exposure” includes short term (acute) exposure to an environment characterised by a high level of aerosolised smoke particles. Exposure may include inhalation and respiration of the aerosolised particles such that the aerosolised particles enter into the respiratory tract of the individual, causing damage. As such, in the context of the present invention, an obstructive pulmonary disease associated with exposure to acute smoke includes obstructive pulmonary diseases which result from (or suspected to be caused by) inhalation (whether passive or active) of smoke particles.

The “individual” or “subject” requiring treatment or prophylaxis in accordance with the present invention includes a mammal. The mammal may be a human, or may be a domestic, zoo, or companion animal. While it is particularly contemplated that the methods of the invention are suitable for medical treatment of humans, they are also applicable to veterinary treatment, including treatment of companion animals such as dogs and cats, and domestic animals such as horses, cattle and sheep, or zoo animals such as felids, canids, bovids, and ungulates.

The skilled person will be familiar with the various methods to identify an individual requiring treatment for obstructive airway disease, including for example a determination of an FEV1/FVC ratio (ratio of forced expiratory volume in 1 second, FEV1 to forced vital capacity, FCV) of less than 0.7 (i.e. the inability to exhales 70% of their breath in one minute). For example, in COPD, there is an increase in airway resistance, shown by a decrease in the forced expiratory volume in 1 second (FEV1) measured by spirometry. COPD is sometimes defined as a forced expiratory volume in 1 second to forced vital capacity ratio (FEV1/FVC) that is less than 0.7. The residual volume, the volume of air left in the lungs following full expiration, is often increased in COPD, as is the total lung capacity, while the vital capacity remains relatively normal. The increased total lung capacity (hyperinflation) can result in the clinical feature of a “barrel chest”—a chest with a large front-to-back diameter that occurs in some individuals with COPD. Hyperinflation can also be seen on a chest x-ray as a flattening of the diaphragm.

The skilled person will be familiar with the various instruments, methods and guidelines available for performing lung function tests. Examples of such guidelines include the GOLD guidelines to determine lung diffusing capacity (Rabe et al., 2007, Am J Respir Crit Care Med, 176:532-555). Spirometry and a peak flow meter can also be used to record variations in airflow limitation.

Other methods for diagnosis of obstructive airway disease include use of chest x-ray (to look for hyperinflation in the case of COPD) or CT scan (to determine presence of emphysema).

The term “therapeutically effective amount” refers to an amount of an AON, capable of treating, preventing or ameliorating obstructive pulmonary disease. A therapeutically effective amount may be determined empirically and in a routine manner in relation to treating obstructive pulmonary conditions.

It will also be appreciated that the present invention includes methods whereby the individual may not be in imminent risk of being exposed to the relevant aerosol, but may have previously been exposed to the aerosol, either for an extended period (long-term, chronic exposure) or for a short period of time (acute exposure). The skilled person will therefore appreciate that there may be a period of time between exposure to the aerosol and the onset of symptoms of obstructive pulmonary disease and the subsequent need for treatment. In some embodiments, the period of time between exposure to the aerosol and treatment is 1 day, 1 week, 1-6 months, 1 year or longer. Preferably, the period of time between exposure to the aerosol and treatment is less than 1 year.

It will be appreciated that some of the above mentioned obstructive pulmonary conditions result in significant destruction to the respiratory system. For example, in the case of emphysema, the skilled person would understand that emphysema is not reversible—once the bronchioles are destroyed, they cannot be replaced. Accordingly, the present invention also contemplates methods of preventing the development of such irreversible damage to the lung tissue.

As used herein, “preventing”, “prevention”, “preventative” or “prophylactic” refers to keeping from occurring, or to hinder, defend from, or protect from the occurrence of a condition, disease, disorder, or phenotype, including an abnormality or symptom. A subject in need of prevention may be prone to develop the condition.

Prevention may include immediate prophylactic steps aimed at reducing any damage to the lung tissue resulting from recent exposure to an aerosol. For example, the present invention includes the use of an AON as a prophylactic for use immediately following smoke exposure, preferably acute or chronic smoke exposure, so to reduce or prevent inflammation. Such prophylaxis may include administration of an AON to an individual immediately following inhalation of smoke from a cigarette (either as the smoker or bystander), administration immediately before a day at work (for those individuals exposed to occupational aerosols), administration immediately before or following an extended stay in a region of poor air quality or industrial pollution (for example, a visit to an industrial city). For example, prophylaxis may also include administration of the AON in the morning, before leaving the house and being exposed to air pollution.

The term “effective to prevent” refers to an amount of nuclease capable of preventing the development of obstructive pulmonary disease.

The skilled person will appreciate that both short and long term approaches can be employed for determining the successful treatment of an individual using the methods of the present invention. Firstly, when considering acute, short term use of an AON to treat an obstructive pulmonary disease as herein defined, the skilled person may look to an immediate improvement of symptoms following treatment. For example, the skilled person would look for signs of improved airflow, reduced airway inflammation, reduced coughing, reduced wheezing, reduced hyperplasia and reduced hypersecretion of mucous glands, reduced mucous production and reduced incidence of productive cough as indicators of successful treatment of an individual using the methods of the present invention.

In addition, the skilled person may perform standard tests to determine whether there is an improvement or decrease in the levels of certain inflammatory markers associated with the presence, or development or risk of obstructive pulmonary disease. Examples of inflammatory markers that would be assessed to determine successful treatment or prophylaxis include measuring levels of C-reactive protein (CRP), serum IgE and IgG, IL-8, sICAM, soluble tumour necrosis factor receptor (STNFR)-1, alpha1-antitrypsin, total white blood cell and neutrophil counts, and percent neutrophils, osteoprotegrin, neutrophil activating peptide-2, CXCL16 and monocyte chemoattractant protein-4. The skilled person would be familiar with methods for measuring levels of these inflammatory markers in an individual receiving treatment in accordance with the present invention, including the use of ELISA, flow cytometry and other methods for measuring inflammatory markers in plasma samples from patients.

Where the methods of the present invention are used over the longer term, including as prophylactic methods, the skilled person would look to improved lung function (as measured by conventional methods described herein) and decreased progression of lung disease as indicators of the successful treatment or prevention of obstructive pulmonary diseases caused by aerosols.

### Smoke

Exposure to acute smoke may refer to transient and intermittent exposure to pulmonary toxins contained in smoke. The pathophysiological pathways that are activated in the lungs following acute exposure to cigarette smoke lead to a progressive decline in lung function. In particular, a key underlying mechanism is airways inflammation which is observed following acute exposure to cigarette smoke in mice, as well as following repeated acute exposure, where it leads to thickening of the bronchial walls and emphysema.

Chronic Obstructive Pulmonary Disease (COPD) is the cumulative result of repeated exposure to inhaled toxins, chiefly cigarette and other types of tobacco and marijuana smoke (e.g. pipe, cigar, water pipe, bidis and kreteks), emissions from heated tobacco products and smokeless tobacco (e.g. vaping) and air pollution (e.g. burning of wood and other biomass fuels, particulate matter, chemical agents, fumes, ground level ozone, oxides of nitrogen or sulfur, heavy metals, passive smoking) that damage the lungs. There is now evidence that most smokers develop some respiratory impairment due to their smoking and experience a faster annual rate of lung function (FEV1) decline than non-smokers. Cigarette smoke induced COPD (COPD-C) is the cumulative result of repeated “acute” exposure to cigarette smoke.

The smoke may be an aerosol or particulate matter from one or more of cigarettes including electronic cigarettes, tobacco or marijuana, coal, wood, oil, gas or other biomass fires. The above are examples of smoke related aerosols which may cause injury or an insult to the lung and include aerosolised carbon particles (which may be found in smoke from burnt organic matter, coal dust, smog, exhaust fumes and other industrial pollutants).

The level of exposure to nicotine containing smoke (such as cigarette smoke) may be determined by measuring levels of cotinine, a metabolite of nicotine, in a saliva, urine, hair or blood sample. Exemplary values of cotinine levels for unexposed nonsmokers, passive smokers and active smokers are provided in the table below.

| | Unexposed | Passive | Active |
|---|---|---|---|
| Matrix | Nonsmokers | Smokers | Smokers |
| Plasma (ng/mL) | 0.09 to 0.7 | 2 to 10 | Greater than 10 |
| Urine (ng/mL) | Less than 10 | 10 to 100 | Greater than 200 |
| Saliva (ng/mL) | 0 to less than 5 | 5 to 10 | Greater than 10 |

Typically, urine cotinine levels in a nonsmoker are generally less than 10 ng/ml, cotinine levels in a light smoker or someone exposed to secondhand smoke are 11 ng/ml to 30 ng/ml and cotinine levels in a heavy smoker may be more than 500 ng/ml Cotinine levels can be determined using standard techniques, including immunoassay techniques, well known in the art, or by using commercially available kits.

The level of exposure to smoke may be determined by measuring levels of blood carboxyhaemoglobin (COHb) levels. For example, an active smoker may have at least about 1.0%, at least about 1.5%, at least about 2%, at least about 2.5%, at least about 3%, at least about 3.5%, at least about 4%, at least about 4.5%, at least about 5%, at least about 6%, at least about 7%, at least about 8%, at least about 9%, or at least about blood 10% COHb levels. Blood carboxyhaemoglobin (COHb) levels can be determined using standard spectrophotometer techniques well known in the art.

Alternatively, the level of exposure to smoke may be determined using a PM2.5 average. For example, during acute smoke exposure the 1 hour PM2.5 average may be from 25 μg/m³ to 50 μg/m³, from 50 μg/m³ to 100 μg/m³, from 100 μg/m³ to 300 μg/m³ or greater than 300 μg/m³. Alternatively, a chronic smoke exposure may have an overall PM2.5 average, for example overall 1 year, of at least 15 μg/m³, at least 20 ug/m³, at least 25 μg/m³, or at least 30 μg/m³.

One of the primary causes of obstructive pulmonary disease, in particular COPD, is inhalation of cigarette smoke. However, the skilled person will appreciate that other types of smoke or insult may also result in obstructive airway disease. Examples of such smoke includes smoke from tobacco in general (eg, from cigars, tobacco pipes) or other “smoked” article (e.g. marijuana joint) which is inhaled by an individual for recreational purposes. It will also be understood that obstructive pulmonary diseases may also result from inhalation of ‘second-hand smoke’ (so-called non-smoking bystanders who inhale the tobacco smoke generated by a smoker). It has also been reported that the risk of COPD in infants is increased if the mother smokes during pregnancy. As used herein a ‘smoker’ is typically an individual who smokes more than 10 packets of cigarettes per year. A non-smoker is someone who typically smokes fewer than 10 packets of cigarettes per year.

Smoke which results in obstructive airway disease can also be smoke from fires, including oil fires, wood fires (e.g. as used in cooking or from bushfires), grass fires, coal fires, tyre fires or biomass fires (such as fires using animal dung as fuel which is used for cooking in developing countries).

In the context of environment pollutants, relevant aerosols include soot, fumes from car exhausts including diesel fumes and smog in industrial communities. For example, individuals living in a highly industrialised region or in a region affected by severe pollution or poor air quality will be at greater risk of developing obstructive pulmonary disease and are candidates for treatment/therapy in accordance with the present invention.

As used here, a “subject that has not substantially reduced their exposure to smoke” refers to a subject that has not substantially or significantly reduced their exposure to a source of smoke. Preferably, the subject has not changed their overall exposure to sources of smoke, in particular cigarette or tobacco smoke or vaping.

A subject in need thereof is one who is, has been, or will be exposed to smoke, preferably acute or chronic exposure to smoke. For example, the subject may be an active or former cigarette smoker, that is currently or has been exposed to second-hand smoke, is currently or has been exposed to wood or forest fire smoke, and/or is currently or has been exposed to gaseous or particulate natural or man-made air pollutants. The subject may not be a cigarette smoker, or smoked less than a pack a day in the previous year, but has been or is being exposed to an acute level of smoke.

Alternatively, the subject is currently, or has had chronic smoke exposure lasting for at least 1 year, at least 2 years, at least 3 years, at least 4 years, at least 5 years, at least 10 years, at least 15 years, at least 20 years, at least 25 years, at least 30 years, at least 35 years, at least 40 years or at least 50 years. For example, the subject may currently be, or has been, a chronic (long-term) cigarette or tobacco smoker or vaper for at least 1 year, at least 2 years, at least 3 years, at least 4 years, at least 5 years, at least 10 years, at least 15 years, at least 20 years, at least 25 years, at least 30 years, at least 35 years, at least 40 years or at least 50 years. In particular, a chronic (long-term) smoker may have smoked a pack a day or more than a pack day in the previous year.

The skilled person will appreciate that in the context of the present invention, there are some individuals who will be more susceptible (sensitive or at risk) to developing obstructive pulmonary disease associated with exposure to an aerosol. For example, such sensitive individuals may be individuals having an underlying condition, such as asthma or other autoimmune disease. Alternatively, an individual at greater risk of obstructive pulmonary disease may be an individual who has received multiple insults to the respiratory tract (for example, extended periods of infection or ill-health).

### RAGE Pre-mRNA Alternate Splicing

Alternative splicing is recognized as an important layer of post-transcriptional gene regulation for the Receptor for Advanced Glycation End-products (RAGE). Although most RAGE is expressed in its full length isoform, a number of different coding isoforms are generated through alternative splicing (also known as splicoforms), including splicoforms with N-terminal truncations, C-terminal truncations, and splicoforms retaining intronic sequences. These different splicoforms may act as possible regulators of the full-length RAGE receptor either by competitive ligand binding or by displacing the full-length protein from binding partners. Over twenty splicoforms have been identified in different tissues such as lung, liver, kidney, smooth muscle, endothelial cells and brain.

The different RAGE gene splice variants have been named RAGE, RAGE_v1 to RAGE_v19 according to the Human Gene Nomenclature Committee and are described in Hudson et al., (2008) The FASEB Journal, 22:1572-1580, the contents of which are incorporated in its entirety.

As used herein, “endogenous soluble RAGE” refers to a polypeptide that lacks any signalling elements and/or the transmembrane domain of full-length RAGE. Endogenous soluble RAGE may be encoded, and subsequently translated, by an alternatively spliced RAGE pre-mRNA resulting in the inclusion of exon 9b and/or exclusion (eg skipping) of exon 10, resulting in a premature stop and the complete loss of the trans-membrane and cytoplasmic domains. For example, endogenous soluble RAGE may be encoded by one or more of RAGE_v1, RAGE_v6, RAGE_v8, RAGE_v9 RAGE_v10, RAGE_v15, RAGE_v18, and RAGE_v19 mRNA, preferably RAGE_v1 . . . . Therefore, it will be appreciated that any AON as described herein that promotes splicing in the RAGE pre-mRNA resulting in the inclusion of exon 9b and/or exclusion (eg skipping) of exon 10 may be used in the methods of the invention to promote the production of “endogenous soluble RAGE” in the respiratory tract and blood of a subject.

For example, a “endogenous soluble RAGE” as described herein may be endogenous secretory RAGE (esRAGE) which is encoded by RAGE_v1 mRNA. esRAGE constitutes ~5% of circulating RAGE in humans. The skipping of exon 10 in esRAGE-type splicing is ascribed to the limitation of intron length in higher eukaryotes. Roughly 45 nucleotides must separate the 5′ splice site and branch point, and the minimum distance between the branch point and 3′ splice site appears to be approximately 18 nucleotides, respectively. Therefore, introns shorter than 70 nucleotides are extremely rare in mammals and cannot be spliced out efficiently. When the esRAGE 5′ splice site in intron 9 is selected, the distance between this site and the 3′ splice site that borders exon 10 is 46 nucleotides, which is considerably shorter than the lower limit of the intron length. Therefore, the use of the downstream, esRAGE 5′ splice site of intron 9 and the inclusion of exon 10 would be mutually exclusive. Among the known splice variants analyzed, all variants that used the downstream esRAGE 5′ splice site in intron 9 skipped exon 10; in contrast, all variants that used the upstream RAGE 5′ splice site in intron 9 included exon 10. Thus, the available evidence indicates that the selection of either one of the two alternative 5′ splice sites in intron 9 couples with inclusion or exclusion of exon 10. The means of regulation of this splicing or an external means to modulate has been previously unknown.

Membrane bound RAGE may be wildtype or full length RAGE, or a protein that has resulted from normal splicing. In other words, membrane bound RAGE contains a transmembrane domain and a short (42 amino acid) cytosolic tail. Further, the membrane bound RAGE may be encoded by a mature RNA, post-splicing, that contains does not include exon 9b and/or that does include exon 10.

### Soluble Splicoforms of Receptor for Advanced Glycation End-Products (RAGE)

Alternative splicing is recognized as an important layer of post-transcriptional gene regulation for the Receptor for Advanced Glycation End-products (RAGE). Although most RAGE is expressed in its full length isoform, a number of different coding isoforms are generated through alternative splicing (also known as splicofoms), including splicoforms with N-terminal truncations, C-terminal truncations, and splicoforms retaining intronic sequences. These different splicoforms may act as possible regulators of the full-length RAGE receptor either by competitive ligand binding or by displacing the full-length protein from binding partners. Over twenty splicoforms have been identified in different tissues such as lung, liver, kidney, smooth muscle, endothelial cells and brain.

The different RAGE gene splice variants have been named RAGE, RAGE_v1 to RAGE_v19 according to the Human Gene Nomenclature Committee and as described in Hudson et al., (2008) The FASEB Journal, 22:1572-1580, the contents of which are incorporated in its entirety. For example, (run on) retention of intron 9 (exon 9b) results in a premature stop and the complete loss of the trans-membrane and cytoplasmic domains generating a C-terminus truncated splicoform. For example, RAGE_v1 is C-terminus truncated splicoform generated by skipping of exon 10 and retention of intron 9 (exon 9b). Alternatively, in another example, an AON as described herein may increase RAGE_v10 mRNA levels in a tissue or sample by promoting splicing in the RAGE pre-mRNA that results in the skipping of exon 10 and exon 11.

The C-terminus truncated splicoforms as described herein lack any signalling elements or the transmembrane domain and translate a c-terminus truncated RAGE, such as endogenous secretory RAGE (esRAGE), that is able to act as a decoy receptor, competing with full length RAGE for ligands or increasing ligand clearance. In particular, esRAGE constitutes ~5% of circulating RAGE in humans.

Higher circulating levels of esRAGE are associated with improved health outcomes and longevity while lower esRAGE is associated with many disease states including but not limited to atherosclerosis, diabetes, the metabolic syndrome, cardiovascular mortality, anaemia, autism and various tumorigenic states. Treatment of diabetic mice with recombinant esRAGE reduces atherosclerosis, vascular inflammation, renal and retinal damage.

Aberrant splicing of RAGE (and therefore dysfunctional RAGE signalling) has been reported in diabetes, some cancers and Alzheimer's disease.

The skipping of exon 10 in esRAGE-type splicing is ascribed to the limitation of intron length in higher eukaryotes. Roughly 45 nucleotides must separate the 5′ splice site and branch point, and the minimum distance between the branch point and 3′ splice site appears to be approximately 18 nucleotides, respectively. Therefore, introns shorter than 70 nucleotides are extremely rare in mammals and cannot be spliced out efficiently. When the esRAGE 5′ splice site in intron 9 is selected, the distance between this site and the 3′ splice site that borders exon 10 is 46 nucleotides, which is considerably shorter than the lower limit of the intron length. Therefore, the use of the downstream, esRAGE 5′ splice site of intron 9 and the inclusion of exon 10 would be mutually exclusive. Among the known splice variants analyzed, all variants that used the downstream esRAGE 5′ splice site in intron 9 skipped exon 10; in contrast, all variants that used the upstream RAGE 5′ splice site in intron 9 included exon 10. Thus, the available evidence indicates that the selection of either one of the two alternative 5′ splice sites in intron 9 couples with inclusion or exclusion of exon 10. The means of regulation of this splicing or an external means to modulate has been previously unknown.

### Antisense Oligonucleotides (AONs)

Antisense oligonucleotide (AON) are short, synthetic, antisense, modified strands of DNA or RNA that can selectively hybridise to pre-RNA/mRNA through Watson-Crick base pairing and selectively modulate the function of the target RNA.

The terms “AON” and “ASO” are both abbreviations of the term “antisense oligonucleotide” and are used interchangeably herein.

Antisense oligonucleotide (AON) are short, synthetic, antisense, modified strands of DNA or RNA that can selectively hybridise to pre-RNA/mRNA through Watson-Crick base pairing and selectively modulate the function of the target RNA.

When AONs are used to modulate alternative splicing of mRNA, they are often referred to as splice-switching oligonucleotides (SSO). In the present invention, the terms AON and SSO may be used interchangeably. SSOs base-pair with a pre-mRNA and disrupt the normal splicing repertoire of the transcript by blocking the RNA-RNA base-pairing or protein-RNA binding interactions that occur between components of the splicing machinery and the pre-mRNA. SSOs can induce “skipping” of selected exons and/or retention of intronic sequences to modulate the product of translation. This can be achieved by targeting splice sites directly or by targeting cis-acting sequences involved in enhancing or silencing splicing by modulating binding of specific proteins or altering secondary structure of the pre mRNA.

Therapeutic SSOs may be used for the treatment of genetic disorders, to skip faulty or misaligned sections allowing for the generation of internally deleted, but now functional protein as a therapy.

The AONs for use in the methods of the present invention selectively manipulate the alternative splicing pattern of RAGE pre-mRNA, resulting in the generation of either natural RAGE mRNA splicoforms that are either non-functional or that act as a decoy receptor to antagonise ligand dependent activation and ligand-independent transactivation of full length RAGE. Preferably, the AONs also reduce the production of membrane bound RAGE.

Notably, there are no common RAGE polymorphisms at these splice sites. The RAGE sequence is highly conserved. Therefore, personalisation or individualised sequence modification is not required, unlike the management of genetic disorders with exon skipping technologies.

According to any aspect of the methods of the invention, the AONs as described herein are capable of binding to a selected target on a RAGE gene transcript to modulate pre-mRNA splicing in a RAGE gene transcript or part thereof. Broadly, the AON may be an isolated or purified AON. By “isolated” is meant material that is substantially or essentially free from components that normally accompany it in its native state. For example, an “isolated polynucleotide” or “isolated oligonucleotide,” as used herein, may refer to a polynucleotide that has been purified or removed from the sequences that flank it in a naturally-occurring state, e.g. a DNA fragment that is removed from the sequences that are adjacent to the fragment in the genome. The term “isolating” as it relates to cells, refers to the purification of cells (e.g. fibroblasts, lymphoblasts) from a source subject (e.g. a subject with a polynucleotide repeat disease). In the context of DNA, mRNA or protein, “isolating” refers to the recovery of the DNA, mRNA or protein from a source, e.g. cells.

According to any aspect of the methods of the invention, the AONs as described herein promote splicing in the RAGE pre-mRNA resulting in the inclusion of exon 9b and/or exclusion (eg skipping) of exon 10, resulting in a premature stop and the complete loss of the trans-membrane and cytoplasmic domains. Therefore, the AONs as described herein may increase the level of RAGE_v1, RAGE_v6, RAGE_v8, RAGE_v9, RAGE_v10, RAGE_v15, RAGE_v18, and RAGE_v19 mRNA, preferably an increase in the level of RAGE_v1, RAGE_v6, RAGE_v8, RAGE_v9, RAGE_v10, RAGE_v15, RAGE_v18, and RAGE_v19 mRNA in a tissue or sample. For example, an AON as described herein may increase RAGE_v1 mRNA levels in a tissue or sample by promoting splicing in the RAGE pre-mRNA that results in the skipping of exon 10 and retention of intron 9 (exon 9b). In another example, an AON as described herein may increase RAGE_v10 mRNA levels in a tissue or sample by promoting splicing in the RAGE pre-mRNA that results in the skipping of exon 10 and exon 11.

An AON can be said to be “directed to” or “targeted against” a target sequence with which it hybridizes. In certain embodiments, the target sequence includes a region including a 3′ or 5′ splice site of a pre-processed mRNA, a branch point, or other sequences involved in the regulation of splicing, including splice enhancers and splice silencers and sites determining the secondary structure of RNA that influence splicing. The target sequence may be within an exon or within an intron or spanning an intron/exon junction.

In any aspect, the antisense oligonucleotide comprises, consists essentially of or consists of a nucleotide sequence at least 80%, 81%, 82%, 83%, 84%, 85%, 86%, 87%, 88%, 89%, 90%, 91%, 92%, 93%, 94%, 95%, 96%, 97%, 98%, 99% or 100% complementary to a target region of exon 10 of RAGE pre-mRNA over the entirety of the antisense oligonucleotide. Preferably, the 5′-most nucleotide of the AON is nucleotide position 88 or 114 of exon 10 or is between nucleotide positions 88 to 114 of exon 10. Preferably, the 5′-most nucleotide of the target region is nucleotide position 88 or 114 of exon 10 or is between nucleotide positions 88 to 114 of exon 10. Typically, the nucleotide position may be identical or equal to SEQ ID NO: 32, where the first nucleotide is position 1.

| | |
|---|---|
| Human RAGE | GCTCTGTGGGAGGATCAGGGCTGGGAACTCT |
| Exon 10 | AGCCCTGGCCCTGGGGATCCTGGGAGGCCTG |
| (SEQ ID NO: 32) | GGGACAGCCGCCCTGCTCATTGGGGTCATCT |
| | TGTGGCAAAGGCGGCAACGCCGAGGAGAGGA |
| | GAG |

In certain embodiments, the AON has sufficient sequence complementarity to a target RNA (i.e. the RNA for which splice site selection is modulated) to block a region of a target RNA (e.g. pre-mRNA) in an effective manner. In exemplary embodiments, such blocking of RAGE pre-mRNA serves to modulate splicing, either by masking a binding site for a splicosomal protein that would otherwise modulate splicing and/or by altering the structure of the targeted RNA. In some embodiments, the target RNA is target pre-mRNA (e.g. RAGE gene pre-mRNA).

An AON having a sufficient sequence complementarity to a target RNA sequence to modulate splicing of the target RNA means that the AON has a sequence sufficient to trigger the masking of a binding site for a native protein that would otherwise modulate splicing and/or alters the three-dimensional structure of the targeted RNA.

Selected AONs can be made shorter, e.g. about 12 bases, or longer, e.g. about 50 bases, and include a small number of mismatches, as long as the sequence is sufficiently complementary to effect splice modulation upon hybridization to the target sequence, and optionally forms with the RNA a heteroduplex having a Tm of 45° C. or greater.

Preferably, the AON is selected from the group comprising SEQ ID NOS: 1-31 and 33 and/or the sequences set forth in any of Tables 1a-1d. More preferably, the AON is SEQ ID NO: 11, 18, 19, or 20.

In certain embodiments, the degree of complementarity between the target sequence and AON is sufficient to form a stable duplex. The region of complementarity of the AONs with the target RNA sequence may be as short as 8-11 bases, but can be 12-15 bases or more, e.g. 10-50 bases, 10-40 bases, 12-30 bases, 12-25 bases, 15-25 bases, 12-20 bases, or 15-20 bases, including all integers in between these ranges. An AON of about 16-17 bases is generally long enough to have a unique complementary sequence. In certain embodiments, a minimum length of complementary bases may be required to achieve the requisite binding Tm, as discussed herein.

In certain embodiments, oligonucleotides as long as 50 bases may be suitable, where at least a minimum number of bases, e.g. 10-12 bases, are complementary to the target sequence. In general, however, facilitated or active uptake in cells is optimized at oligonucleotide lengths of less than about 30 bases. For phosphorodiamidate morpholino oligomer (PMO) AONs described further herein, an optimum balance of binding stability and uptake generally occurs at lengths of 18-25 bases. Included are AONs (e.g. PMOs, PMO-X, PNAs, LNAs, TINA, 2′-OMe) that consist of about 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 bases.

In certain embodiments, AONs may be 100% complementary to the target sequence, or may include mismatches, e.g. to accommodate variants, as long as a heteroduplex formed between the oligonucleotide and target sequence is sufficiently stable to withstand the action of cellular nucleases and other modes of degradation which may occur in vivo. Hence, certain oligonucleotides may have about or at least about 70% sequence complementarity, e.g. 70%, 71%, 72%, 73%, 74%, 75%, 76%, 92%, 93%, 94%, 95%, 96%, 97%, 98%, 99% or 100% sequence complementarity, between the oligonucleotide and the target sequence.

Mismatches, if present, are typically less destabilizing toward the end regions of the hybrid duplex than in the middle. The number of mismatches allowed will depend on the length of the oligonucleotide, the percentage of G:C base pairs in the duplex, and the position of the mismatch(es) in the duplex, according to well understood principles of duplex stability. Although such an AON is not necessarily 100% complementary to the target sequence, it is effective to stably and specifically bind to the target sequence, such that splicing of the target pre-RNA is modulated.

The stability of the duplex formed between an AON and a target sequence is a function of the binding Tm and the susceptibility of the duplex to cellular enzymatic cleavage. The Tm of an oligonucleotide with respect to complementary-sequence RNA may be measured by conventional methods, such as those described by Hames et al., Nucleic Acid Hybridization, IRL Press, 1985, pp. 107-108 or as described in Miyada C. G. and Wallace R. B., 1987, Oligonucleotide Hybridization Techniques, Methods Enzymol. Vol. 154 pp. 94-107. In certain embodiments, AONs may have a binding Tm, with respect to a complementary-sequence RNA, of greater than body temperature and preferably greater than about 45° C. or 50° C. Tm's in the range 60-80° C. or greater are also included.

Additional examples of variants include AONs having about or at least about 70% sequence identity or homology, e.g. 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% sequence identity or homology, over the entire length of any of SEQ ID NOS: 1-31 and 33 and/or the sequences set forth in any of Tables 1a-1d. More preferably, the AON is SEQ ID NO: 11, 18, 19, or 20.

The modification of pre-mRNA splicing preferably induces “skipping”, or the removal of one or more exons or retention of introns of the mRNA. The resultant protein is preferably of a shorter length when compared to the parent full-length RAGE protein due to either internal truncation or premature termination. Preferably, the resultant protein has a C-terminal truncation. These truncated RAGE proteins may be termed splicoforms of the full length RAGE protein.

The remaining exons of the mRNA generated may be in-frame and produce a shorter protein with a sequence that is similar to that of the parent full length protein, except that it has an internal truncation in a region between the original 3′ and 5′ ends. In another possibility, the exon skipping may induce a frame shift that results in a protein wherein the first part of the protein is substantially identical to the parent full length protein, but wherein the second part of the protein has a different sequence (e.g. a nonsense sequence) due to a frame-shift. Alternatively, the exon skipping may induce the production of a prematurely terminated protein due to a disruption of the reading frame and presence of a premature termination of translation. The prematurely terminated protein may be the result of mRNA that is prematurely terminated (e.g. skipping of exons 10 and/or 11) or may be the result of a run on into an intron (e.g. RAGE 9b) or missense skip which provides an mRNA that contains the exon 10 and/or 11 mRNA, but which does not provide expression of the protein encoded by these exons.

Skipping individual exons of exons 1 to 9 will preferably disrupt the reading frame of the RAGE transcript. This will lead to increased degradation of RNA through nonsense mediated decay.

Skipping individual exons of exons 1 to 11 will preferably keep the reading frame intact. This will preferably lead to translation into an internally truncated protein. The truncated protein or RAGE mRNA splicoform may have a completely ablated function, may have a reduced function or act as a decoy receptor.

Preferably, these truncated, nonsense or prematurely terminated proteins are lacking one or more functional domains involved the induction of intracellular signalling pathways by RAGE ligands or non-ligand-dependent transactivation of RAGE by collocated GPCRs. For example, Exon 10 encodes a transmembrane domain and removing this exon may generate a soluble RAGE protein, which could potentially act as a soluble decoy or competitive antagonist of ligand induced signalling via RAGE. Truncated, nonsense or prematurely terminated proteins may further lack an attachment or binding site for other factors, removal of which may lead to a reduction in interaction of the RAGE protein with relevant signalling pathways.

Alternatively, the removal of one or more exons may lead to misfolding of the RAGE protein and a reduction in the ability of the protein to be successfully transported through the membrane.

The presence of internally truncated proteins (i.e. proteins lacking the amino acids encoded by one or more exons) is preferable. If the RAGE protein is inhibited, there may be problems with elevation of RAGE transcription as the body tries to compensate for the reduction in the total amount of RAGE protein. In contrast, the presence of an internally truncated protein (preferably lacking one or more of the features of the complete RAGE protein), should be sufficient to prevent elevated transcription, but still provide a therapeutic advantage due to a reduction in the total amount of functional RAGE protein.

The AON induced exon skipping as described herein need not completely or even substantially ablate the function of the RAGE protein. Preferably, the modulation of alternative splicing via the exon skipping process results in a reduced or compromised functionality of the RAGE protein.

The different isoforms of RAGE produced using different skipping strategies could result in proteins with ablated or reduced signalling activity that could preferably be used to treat or prevent different diseases associated with RAGE activity, such as neurodegenerative diseases, cancer, lung disorders, or inflammatory diseases. Alternative splicing strategies may form truncated proteins or proteins with reduced functions that can be preferably used as treatments for specific aspects, forms or progression of the diseases associated with RAGE expression and activity.

The skipping process using AONs may exclude (skip) an individual exon, or may result in skipping two or more exons at once.

The skipping process using AONs may include retention of intronic sequences with or without directly skipping one or more exons.

The AONs for use in the methods of the present invention may be a combination of two or more AONs capable of binding to a selected target to induce exon exclusion in a RAGE gene transcript. The combination may be a cocktail of two or more AONs and/or a construct comprising two or more or two or more AONs joined together.

TABLE 1a
Sequence of AONs for modulation of alternative splicing in human RAGE
Exon 9

| SEQ ID NO | AON# | Name | Sequence (5′ to 3′) |
|---|---|---|---|
| 1 | 3668 | H-AGERex9A(−10+15) | CCUCCUCGCCUGGUUCUGGAAGACA |
| 2 | 3669 | H-AGERex9A(+02+21) | UUGGCCCCUCCUCGCCUGGU |
| 3 | 3670 | H-AGERex9A(+08+27) | CUGCAGUUGGCCCCUCCUCG |
| 4 | 3671 | H-AGERex9D(+07−13) | UCAAACCCCUCACCUGCAGU |
| 5 | 4103 | H-AGERex9A(+3+27) | CCUGCAGUUGGCCCCUCCUCGCCUG |
| 6 | 4104 | H-AGERex9A(+10+27) | CCCCUCACCUGCAGUUGGCCCCUCC |
| 7 | 4105 | H-AGERex9A(+8+25) | GCAGUUGGCCCCUCCUC |
| 8 | 4106 | H-AGERex9D(+22-3) | CACCUGCAG UUG GCCCCUCCUCGCC |

TABLE 1b
Sequence of AONs for modulation of alternative splicing in human RAGE
Exon 10

| SEQ ID NO | AON# | Name | Sequence (5′ to 3′) |
|---|---|---|---|
| 9 | 3777 | AGER_H10A(−10+15) | AUCCUCCCACAGAGCCUGUACGGAG |
| 10 | 3778 | AGER_H10A(−5+20) | CCCUGAUCCUCCCACAGAGCCUGUA |
| 11 | 3779 | AGER_H10A(+90+114) | GGCGUUGCCGCCUUUGCCACAAGAU |
| 12 | 3780 | AGER_H10D(+20−5) | CUCACCUCUCCUCUCCUCGGCGUUG |
| 13 | 3781 | AGER_H10D(+15−10) | CUCCACUCACCUCUCCUCUCCUCGG |
| 14 | 82 | AGER_H10A(+60+79) | AGCAGGGCGGCUGUCCCCAG |
| 15 | 83 | AGER_H10A(+75+99) | GCCACAAGAUGACCCCAAUGAGCAG |
| 16 | 84 | AGER_A10(+80+104) | CCUUUGCCACAAGAUGACCCCAAUG |
| 17 | 85 | AGER_A10(+85+109) | UGCCGCCUUUGCCACAAGAUGACCC |
| 18 | 87 | AGER_H10(+95+119) | UCCUCGGCGUUGCCGCCUUUGCCAC |
| 19 | 90 | AGER_H10A(+90+102) | UUUGCCACAAGAU |
| 20 | 93 | AGER_H10A(+88+107) | CCGCCUUUGCCACAAGAUGA |

TABLE 1c
Sequence of AONs for modulation of alternative splicing in human RAGE
Intron 9

| SEQ ID NO | AON# | Name | Sequence (5′ to 3′) |
|---|---|---|---|
| 21 | 88 | AGER_H10A(−75−51) | UUUCUUGUUGACCAUCCCCCCAGUC |
| 22 | 89 | AGER_H10A(−86−62) | CCAUCCCCCCAGUCACAUGUGUUGG |

TABLE 1d
Sequence of AONs for modulation of alternative splicing in murine RAGE

| SEQ ID NO | AON# | Name | Sequence (5′ to 3′) |
|---|---|---|---|
| 23 | 3672 | M-AgerEx9A(−10+15) | CCUCCUCGCCUGGUUCUGGAAGACA |
| 24 | 3673 | M-AgerEx9A(+02+21) | CUGGCCCCUCAUCGCCGGUU |
| 25 | 3674 | M-AgerEx9A(+08+27) | CUUCAGCUGGCCCCUCAUCG |
| 26 | 3675 | M-AgerEx9D(+07−13) | UCCAGUCCCUCACCUUCAGC |
| 27 | m3779 | M-AgerEx10A(+90+114) | UGGGUUGUCGUUUUCGCCACAGGAU |
| | (m79) | | |
| 28 | m97 | M-AgerEx10A(+48,+70) | ACUACUCCCAGGCCUCCCAGGAU |
| 29 | m98 | M-AgerEx10A(+56+79) | GCAGGGCUACUACUCCCAGGCA |
| 30 | m101 | M-AgerEx10D(−15+10) | UCUCACUCACCUCUCCUCACGCCUG |
| 31 | m102 | M-AgerEx10A(−5+20) | CCCAGACUCACCCACAGAGCCUGUA |
| 33 | 6713 | M-AgerEx10A(+90+107) | TCGTTTTCGCCACAGGAT |

More specifically, the AON for use in the methods of the present invention may be selected from those set forth in any of Tables 1a-1d. The sequences are preferably selected from the group consisting of any one or more of any one or more of SEQ ID NOs: 1-31 and 33, and combinations or cocktails thereof. More preferably, the AON is SEQ ID NO: 11, 18, 19, or 20. The combination of AONs is preferably a combination of SEQ ID NO: 11 and 10, or SEQ ID NO: 11 and 13. This includes sequences which can hybridise to such sequences under stringent hybridisation conditions, sequences complementary thereto, sequences containing modified bases, modified backbones, and functional truncations or extensions thereof which possess or modulate pre-mRNA processing activity in a RAGE gene transcript.

In the context of the AONs for use in the methods of the present invention, those skilled in the art will recognise that both U and T residues are capable of binding A and are therefore interchangeable in any of the AON sequences disclosed herein. Accordingly, in any AON sequence disclosed herein, any T may be a U and/or any U may be a T. These U to T or T to U substitutions produce equally potent AONs for use in the methods of the present invention.

The oligomer and the DNA, cDNA or RNA are complementary to each other when a sufficient number of corresponding positions in each molecule are occupied by nucleotides which can hydrogen bond with each other. Thus, “specifically hybridisable” and “complementary” are terms which are used to indicate a sufficient degree of complementarity or pairing such that stable and specific binding occurs between the oligomer and the DNA, cDNA or RNA target. It is understood in the art that the sequence of an AON need not be 100% complementary to that of its target sequence to be specifically hybridisable. An AON is specifically hybridisable when binding of the compound to the target DNA or RNA molecule interferes with the normal function of the target DNA or RNA product, and there is a sufficient degree of complementarity to avoid non-specific binding of the AON to non-target sequences under conditions in which specific binding is desired, i.e. under physiological conditions in the case of in vivo assays or therapeutic treatment, and in the case of in vitro assays, under conditions in which the assays are performed.

Selective hybridisation may be under low, moderate or high stringency conditions, but is preferably under high stringency. Those skilled in the art will recognise that the stringency of hybridisation will be affected by such conditions as salt concentration, temperature, or organic solvents, in addition to the base composition, length of the complementary strands and the number of nucleotide base mismatches between the hybridising nucleic acids. Stringent temperature conditions will generally include temperatures in excess of 30° C., typically in excess of 37° C., and preferably in excess of 45° C., preferably at least 50° C., and typically 60° C.-80° C. or higher. Stringent salt conditions will ordinarily be less than 1000 mM, typically less than 500 mM, and preferably less than 200 mM. However, the combination of parameters is much more important than the measure of any single parameter. An example of stringent hybridisation conditions is 65° C. and 0.1×SSC (1×SSC=0.15 M NaCl, 0.015 M sodium citrate pH 7.0). Thus, the AONs for use in the methods of the present invention may include oligomers that selectively hybridise to the sequences provided in any of Tables 1a-1d, or SEQ ID NOs: 1-31 and 33. More preferably, the AON is SEQ ID NO: 11, 18, 19, or 20.

It will be appreciated that the codon arrangements at the end of exons in structural proteins may not always break at the end of a codon, consequently there may be a need to delete more than one exon from the pre-mRNA to ensure in-frame reading of the mRNA. In such circumstances, a plurality of AONs may need to be selected by the method of the invention wherein each is directed to a different region responsible for inducing inclusion of the desired exon and/or intron. At a given ionic strength and pH, the Tm is the temperature at which 50% of a target sequence hybridizes to a complementary polynucleotide. Such hybridization may occur with “near” or “substantial” complementarity of the AON to the target sequence, as well as with exact complementarity.

Typically, selective hybridisation will occur when there is at least about 55% identity over a stretch of at least about 14 nucleotides, preferably at least about 65%, more preferably at least about 75% and most preferably at least about 90%, 95%, 98% or 99% identity with the nucleotides of the AON. The length of homology comparison, as described, may be over longer stretches and in certain embodiments will often be over a stretch of at least about nine nucleotides, usually at least about 12 nucleotides, more usually at least about 20, often at least about 21, 22, 23 or 24 nucleotides, at least about 25, 26, 27 or 28 nucleotides, at least about 29, 30, 31 or 32 nucleotides, at least about 36 or more nucleotides.

Thus, the AON for use in the methods of the present invention sequences preferably have at least 75%, more preferably at least 85%, more preferably at least 86, 87, 88, 89 or 90% homology to the sequences shown in the sequence listings herein. More preferably there is at least 91, 92, 93 94, or 95%, more preferably at least 96, 97, 98% or 99%, homology. Generally, the shorter the length of the AON, the greater the homology required to obtain selective hybridisation. Consequently, where an AON consists of less than about 30 nucleotides, it is preferred that the percentage identity is greater than 75%, preferably greater than 85, 86, 87, 88, 89, 90, 91, 92, 93, 94, 95%, 96, 97, 98% or 99% compared with the AONs set out in the sequence listings herein. Nucleotide homology comparisons may be conducted by sequence comparison programs such as the GCG Wisconsin Bestfit program or GAP (Deveraux et al., 1984, Nucleic Acids Research 12, 387-395). In this way sequences of a similar or substantially different length to those cited herein could be compared by insertion of gaps into the alignment, such gaps being determined, for example, by the comparison algorithm used by GAP.

The AONs for use in the methods of the present invention may have regions of reduced homology, and regions of exact homology with the target sequence. It is not necessary for an oligomer to have exact homology for its entire length. For example, the oligomer may have continuous stretches of at least 4 or 5 bases that are identical to the target sequence, preferably continuous stretches of at least 6 or 7 bases that are identical to the target sequence, more preferably continuous stretches of at least 8 or 9 bases that are identical to the target sequence. The oligomer may have stretches of at least 10, 11, 12, 13, 14, 15, 16, 17, 18, 19, 20, 21, 22, 23, 24, 25 or 26 bases that are identical to the target sequence. The remaining stretches of oligomer sequence may be intermittently identical with the target sequence; for example, the remaining sequence may have an identical base, followed by a non-identical base, followed by an identical base. Alternatively (or as well) the oligomer sequence may have several stretches of identical sequence (for example 3, 4, 5 or 6 bases) interspersed with stretches of less than perfect homology. Such sequence mismatches will preferably have no or very little loss of splice switching activity.

The term “modulate” or “modulates” includes to “increase” or “decrease” one or more quantifiable parameters, optionally by a defined and/or statistically significant amount. The terms “increase” or “increasing,” “enhance” or “enhancing,” or “stimulate” or “stimulating” refer generally to the ability of one or AONs or compositions to produce or cause a greater physiological response (i.e. downstream effects) in a cell or a subject relative to the response caused by either no AON or a control compound. The terms “decreasing” or “decrease” refer generally to the ability of one or AONs or compositions to produce or cause a reduced physiological response (i.e. downstream effects) in a cell or a subject relative to the response caused by either no AON or a control compound.

Relevant physiological or cellular responses (in vivo or in vitro) will be apparent to persons skilled in the art, and may include increases in the exclusion of specific exons in a RAGE-coding pre-mRNA, decreases in the amount of RAGE-coding pre-mRNA or decreases in the expression of functional RAGE protein in a cell, tissue, or subject in need thereof. An “increased” or “enhanced” amount is typically a statistically significant amount, and may include an increase that is 1.1, 1.2, 2, 3, 4, 5, 6, 7, 8, 9, 10, 15, 20, 30, 40, 50 or more times (e.g. 500, 1000 times) (including all integers and decimal points in between and above 1, e.g. 1.5, 1.6, 1.7, 1.8) the amount produced by no AON (the absence of an agent) or a control compound. The term “reduce” or “inhibit” may relate generally to the ability of one or more AONs or compositions to “decrease” a relevant physiological or cellular response, such as a symptom of a disease or condition described herein, as measured according to routine techniques in the diagnostic art. Relevant physiological or cellular responses (in vivo or in vitro) will be apparent to persons skilled in the art, and may include reductions in the symptoms or pathology of a disease such as cancer, neurodegenerative diseases, lung disorders, and other inflammatory diseases. A “decrease” in a response may be statistically significant as compared to the response produced by no AON or a control composition, and may include a 1%, 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, 50%, 55%, 60%, 65%, 70%, 75%, 80%, 85%, 90%, 95%, or 100% decrease, including all integers in between.

The length of an AON may vary, as long as it is capable of binding selectively to the intended location within the pre-mRNA molecule. The length of such sequences can be determined in accordance with selection procedures described herein. Generally, the AON will be from about 10 nucleotides in length, up to about 50 nucleotides in length. It will be appreciated, however, that any length of nucleotides within this range may be used in the method. Preferably, the length of the AON is between 10 and 40, 10 and 35, 15 to 30 nucleotides in length or 20 to 30 nucleotides in length, most preferably about 25 to 30 nucleotides in length. For example, the oligomer may be 20, 21, 22, 23, 24, 25, 26, 27, 28, 29 or 30 nucleotides in length.

As used herein, an “AON” refers to a linear sequence of nucleotides, or nucleotide analogs, that allows the nucleobase to hybridize to a target sequence in an RNA by Watson-Crick base pairing, to form an oligonucleotide: RNA heteroduplex within the target sequence. The terms “AON”, “AON”, “oligomer” and “antisense compound” may be used interchangeably to refer to an oligonucleotide. The cyclic subunits may be based on ribose or another pentose sugar or, in certain embodiments, a morpholino group (see description of morpholino oligonucleotides below). Also contemplated are peptide nucleic acids (PNAs), locked nucleic acids (LNAs), and 2′-O-Methyl oligonucleotides, 2′-O-Methoxyethyl oligonucleotides, among other antisense agents known in the art.

Included are non-naturally-occurring AONs, or “oligonucleotide analogs”, including AONs or oligonucleotides having (i) a modified backbone structure, e.g. a backbone other than the standard phosphodiester linkage found in naturally-occurring oligo- and polynucleotides, and/or (ii) modified sugar moieties, e.g. morpholino moieties rather than ribose or deoxyribose moieties. Oligonucleotide analogs support bases capable of hydrogen bonding by Watson-Crick base pairing to standard polynucleotide bases, where the analog backbone presents the bases in a manner to permit such hydrogen bonding in a sequence-specific fashion between the oligonucleotide analog molecule and bases in a standard polynucleotide (e.g. single-stranded RNA or single-stranded DNA). Preferred analogs are those having a substantially uncharged, phosphorus containing backbone.

One method for producing AONs is the methylation of the 2′ hydroxyribose position and the incorporation of a phosphorothioate backbone produces molecules that superficially resemble RNA but that are much more resistant to nuclease degradation, although persons skilled in the art of the methods of the invention will be aware of other forms of suitable backbones that may be useable in the objectives of the methods of the invention.

Increased splice-switching may also be achieved with alternative oligonucleotide chemistry. For example, the AON may be chosen from the list comprising: phosphoramidate or phosphorodiamidate morpholino oligomer (PMO); PMO-X; PPMO; peptide nucleic acid (PNA); a locked nucleic acid (LNA) and derivatives including alpha-L-LNA, 2′-amino LNA, 4′-methyl LNA and 4′-O-methyl LNA; ethylene bridged nucleic acids (ENA) and their derivatives; phosphorothioate oligomer; tricyclo-DNA oligomer (tcDNA); tricyclophosphorothioate oligomer; 2′O-Methyl-modified oligomer (2′-OMe); 2′-O-methoxy ethyl(2′-MOE); 2′-fluoro, 2′-fluroarabino (FANA); unlocked nucleic acid (UNA); thermostable twisted intercalating nucleic acid (TINA), hexitol nucleic acid (HNA); cyclohexenyl nucleic acid (CeNA); 2′-amino (2′-NH2); 2′-O-ethyleneamine or any combination of the foregoing as mixmers or as gapmers. To further improve the delivery efficacy, the above mentioned modified nucleotides are often conjugated with fatty acids/lipid/cholesterol/amino acids/carbohydrates/polysaccharides/nanoparticles etc. to the sugar or nucleobase moieties. These conjugated nucleotide derivatives can also be used to construct exon skipping AONs. Antisense oligonucleotide-induced splice modification of the human RAGE gene transcripts have generally used either oligoribonucleotides, PNAs, 20Me or MOE modified bases on a phosphorothioate backbone. When alternative chemistries are used to generate the AONs for use in the methods of the invention, the uracil (U) of the sequences provided herein may be replaced by a thymine (T).

Included within the AONs for use in the methods of the present invention are non-naturally-occurring oligomers, or “oligonucleotide analogues,” including oligomers having (i) a modified backbone structure, e.g. a backbone other than the standard phosphodiester linkage found in naturally-occurring oligo- and polynucleotides, and/or (ii) modified sugar moieties, e.g. morpholino moieties rather than ribose or deoxyribose moieties. Oligomer analogues support bases capable of hydrogen bonding by Watson-Crick base pairing to standard polynucleotide bases, where the analogue backbone presents the bases in a manner to permit such hydrogen bonding in a sequence-specific fashion between the oligomer analogue molecule and bases in a standard polynucleotide (e.g. single-stranded RNA or single-stranded DNA). Preferred analogues are those having a substantially uncharged, phosphorus containing backbone.

Antisense oligonucleotides that do not activate RNase H can be made in accordance with known techniques (see, e.g. U.S. Pat. No. 5,149,797). Such AONs, which may be deoxyribonucleotide or ribonucleotide sequences, simply contain any structural modification which sterically hinders or prevents binding of RNase H to a duplex molecule containing the oligomer as one member thereof, which structural modification does not substantially hinder or disrupt duplex formation. Because the portions of the oligomer involved in duplex formation are substantially different from those portions involved in RNase H binding thereto, numerous AONs that do not activate RNase H are available. For example, such AONs may be oligomers wherein at least one, or all, of the inter-nucleotide bridging phosphate residues are modified phosphates, such as methyl phosphonates, methyl phosphorothioates, phosphoromorpholidates, phosphoropiperazidates boranophosphates, amide linkages and phosphoramidates. For example, every other one of the internucleotide bridging phosphate residues may be modified as described. In another non-limiting example, such AONs are molecules wherein at least one, or all, of the nucleotides contain a 2′ lower alkyl moiety (such as, for example, C₁-C₄, linear or branched, saturated or unsaturated alkyl, such as methyl, ethyl, ethenyl, propyl, 1-propenyl, 2-propenyl, and isopropyl). For example, every other one of the nucleotides may be modified as described.

Specific examples of preferred AONs for use in the methods of the present invention include oligomers containing modified backbones or non-natural inter-nucleoside linkages. As defined in this specification, oligomers having modified backbones include those that retain a phosphorus atom in the backbone and those that do not have a phosphorus atom in the backbone. For the purposes of this specification, and as sometimes referenced in the art, modified oligomers that do not have a phosphorus atom in their inter-nucleoside backbone can also be considered to be AONs.

In other preferred oligomer mimetics, both the sugar and the inter-nucleoside linkage, i.e. the backbone, of the nucleotide units are replaced with novel groups. The base units are maintained for hybridization with an appropriate nucleic acid target compound. One such oligomeric compound, an oligomer mimetic that has been shown to have excellent hybridization properties, is referred to as a peptide nucleic acid (PNA). In PNA compounds, the sugar-backbone of an oligomer is replaced with an amide containing backbone, in particular an aminoethylglycine backbone. The nucleo-bases are retained and are bound directly or indirectly to aza nitrogen atoms of the amide portion of the backbone.

Another preferred chemistry is the phosphorodiamidate morpholino oligomer (PMO) oligomeric compounds, which are not degraded by any known nuclease or protease. These compounds are uncharged, do not activate RNase H activity when bound to a RNA strand and have been shown to exert sustained splice modulation after in vivo administration (Summerton and Weller, Antisense Nucleic Acid Drug Development, 7, 187-197).

Modified oligomers may also contain one or more substituted sugar moieties. Oligomers may also include nucleobase (often referred to in the art simply as “base”) modifications or substitutions. Certain nucleobases are particularly useful for increasing the binding affinity of the oligomeric compounds as described herein. These include 5-substituted pyrimidines, 6-azapyrimidines, and N-2, N-6 and O-6 substituted purines, including 2-aminopropyladenine, 5-propynyluracil, 5-propynylcytosine, and 5-methylcytosine substitutions have been shown to increase nucleic acid duplex stability by 0.6-1.2° C., even more particularly when combined with 2′-O-methoxyethyl sugar modifications.

The activity of AONs and variants thereof can be assayed according to routine techniques in the art. For example, splice forms and expression levels of surveyed RNAs and proteins may be assessed by any of a wide variety of well-known methods for detecting splice forms and/or expression of a transcribed nucleic acid or protein. Non-limiting examples of such methods include RT-PCR of spliced forms of RNA followed by size separation of PCR products, nucleic acid hybridization methods e.g. Northern blots and/or use of nucleic acid arrays; nucleic acid amplification methods; immunological methods for detection of proteins; protein purification methods; and protein function or activity assays.

RNA expression levels can be assessed by preparing mRNA/cDNA (i.e. a transcribed polynucleotide) from a cell, tissue or organism, and by hybridizing the mRNA/cDNA with a reference polynucleotide, which is a complement of the assayed nucleic acid, or a fragment thereof. cDNA can, optionally, be amplified using any of a variety of polymerase chain reaction or in vitro transcription methods prior to hybridization with the complementary polynucleotide; preferably, it is not amplified. Expression of one or more transcripts can also be detected using quantitative PCR to assess the level of expression of the transcript(s).

The AONs for use in the methods of the present invention may provide induced splice-switching of the RAGE gene transcript, clinically relevant oligomer chemistries and delivery systems to direct RAGE splice manipulation to therapeutic levels, promotion of the non-signalling decoy-receptor RAGE mRNA splicoform (e.g. RAGE_v1, RAGE_v6, RAGE_v8, RAGE_v9, RAGE_v10, RAGE_v15, RAGE_v18, and RAGE_v19 mRNA) can be achieved, and therefore may be used to treat a RAGE-related respiratory (pulmonary) disease or disorder.

The AONs for use in the methods of the present invention may be conveniently made through the well-known technique of solid phase synthesis. Equipment for such synthesis is sold by several vendors including, for example, Applied Biosystems (Foster City, Calif.). One method for synthesising oligomers on a modified solid support is described in U.S. Pat. No. 4,458,066.

Any other means for such synthesis known in the art may additionally or alternatively be employed. It is well known to use similar techniques to prepare oligomers such as the phosphorothioates and alkylated derivatives. In one such automated embodiment, diethyl-phosphoramidites are used as starting materials and may be synthesized as described by Beaucage, et al., (1981) Tetrahedron Letters, 22:1859-1862.

The AONs for use in the methods of the present invention are synthesised in vitro and do not include antisense compositions of biological origin, or genetic vector constructs designed to direct the in vivo synthesis of AONs.

### Administration, Dosage and Formulation

In any aspect, the methods of the present invention may be used to prevent or treat a disease associated with, or caused by, smoke exposure. Preferably, the disease associated with, or caused by, acute or chronic smoke exposure.

The term ‘respiratory’ refers to the process by which oxygen is taken into the body and carbon dioxide is discharged, through the bodily system including the nose, throat, larynx, trachea, bronchi and lungs.

As used herein, the upper respiratory tract may include the following regions: nose and nasal passages, paranasal sinuses, the pharynx, and the portion of the larynx above the vocal folds (cords). Typically, the lower respiratory tract includes any one of more of the following regions: portion of the larynx below the vocal folds, trachea, bronchi and bronchioles. The lungs can be included in the lower respiratory tract and include the respiratory bronchioles, alveolar ducts, alveolar sacs, and alveoli.

The term ‘respiratory disease’ or ‘respiratory condition’ refers to any one of several ailments that involve inflammation and affect a component of the respiratory system including the upper (including the nasal cavity, pharynx and larynx) and lower respiratory tract (including trachea, bronchi and lungs).

A symptom of respiratory disease may include cough, excess sputum production, a sense of breathlessness or chest tightness with audible wheeze. Exercise capacity may be quite limited. In asthma the FEV1.0 (forced expiratory volume in one second) as a percentage of that predicted nomographically based on weight, height and age, may be decreased as may the peak expiratory flow rate in a forced expiration. In COPD the FEV1.0 as a ratio of the FVC is typically reduced to less than 0.7. The impact of each of these conditions may also be measured by days of lost work/school, disturbed sleep, requirement for bronchodilator drugs, requirement for glucocorticoids including oral glucocorticoids.

The existence of, improvement in, progression of, treatment of or prevention of a respiratory disease may be determined by any clinically or biochemically relevant method of the subject or a biopsy therefrom. For example, a parameter measured may be the presence or degree of lung function, signs and symptoms of obstruction; exercise tolerance; night time awakenings; days lost to school or work; bronchodilator usage; inhaled corticosteroid (ICS) dose; oral (glucocorticoid) GC usage; need for other medications; need for medical treatment; hospital admission.

As used herein, ‘preventing’ or ‘prevention’ is intended to refer to at least the reduction of likelihood of the risk of (or susceptibility to) acquiring a disease or disorder (i.e. causing at least one of the clinical symptoms of the disease not to develop in a patient that may be exposed to or predisposed to the disease but does not yet experience or display symptoms of the disease). Biological and physiological parameters for identifying such patients are provided herein and are also well known by physicians.

The terms “treatment” or “treating” of a subject includes the application or administration of an AON as described herein with the purpose of delaying, slowing, stabilizing, curing, healing, alleviating, relieving, altering, remedying, less worsening, ameliorating, improving, or affecting the disease or condition, the symptom of the disease or condition, or the risk of (or susceptibility to) the disease or condition. The term “treating” refers to any indication of success in the treatment or amelioration of an injury, pathology or condition, including any objective or subjective parameter such as abatement; remission; lessening of the rate of worsening; lessening severity of the disease; stabilization, diminishing of symptoms or making the injury, pathology or condition more tolerable to the subject; slowing in the rate of degeneration or decline; making the final point of degeneration less debilitating; or improving a subject's physical or mental well-being.

The term “ameliorate” or “amelioration” refers to a decrease, reduction or elimination of a condition, disease, disorder, or phenotype, including an abnormality or symptom. A subject in need of treatment may already have the condition, or may be prone to have the condition or may be one in whom the condition is to be prevented.

The AONs as described herein may be are adapted to aid in the prophylactic or therapeutic treatment, prevention or amelioration of symptoms of a disease such as a RAGE expression related disease or pathology in a form suitable for delivery to a patient.

A positive response to therapy may also be prevention or attenuation of worsening of respiratory symptoms, e.g. asthma symptoms, following smoke exposure. This could be assessed by comparison of the mean change in disease score from baseline to end of study period based on Juniper Asthma Control Questionnaire (ACQ-6), and could also assess lower respiratory symptom score (LRSS—symptoms of chest tightness, wheeze, shortness or breath and cough). Change from baseline lung function (peak expiratory flow PEF) could also be assessed and a positive response to therapy could be a significant attenuation in reduced PEF. For example, a placebo treated group would show a significant reduction in morning PEF of 15% at the peak whilst the treatment group would show a non-significant reduction in PEF less than 15% change from baseline.

Although the methods of the present invention finds application in humans, the methods of the invention is also useful for therapeutic veterinary purposes. The invention is useful for domestic or farm animals such as cattle, sheep, horses and poultry; for companion animals such as cats and dogs; and for zoo animals.

In certain embodiments, the AONs as described herein can be delivered by pulmonary or nasal routes (e.g. via nebulised saline incorporating the AONs). The highest endogenous expression of RAGE mRNA in the healthy human tissues is found in the lung and is accessible via the airways. Inhaled oligonucleotides are an emerging therapeutic modality for respiratory diseases. The airways are uniquely lined with pulmonary surfactants, which are primarily composed of zwitterionic lipids. These surfactant lipids possess cationic properties at the pH of the respiratory tract. When anionic oligonucleotides are inhaled, they tend to be adsorbed by the surfactants, resulting in reformulated particles that have been hypothesised to be efficiently taken up by bronchial and alveolar epithelial cells into the pulmonary cells. Of note, AONs have been shown to be able to withstand the nebulization process.

The AONs as described herein may be in compositions formulated for administration to the lower respiratory tract only. Limitation to the lower respiratory tract may be achieved by an amount, particularly volume and composition of form i.e. particle size, physical form whether dry powder or solution droplet, of composition that would otherwise be administered to the upper respiratory tract. Alternatively, the AONs as described herein may be administered via a device that ensures retention in the lower respiratory tract only.

The AONs as described herein may be formulated for intranasal administration, including dry powder, sprays, mists, or aerosols. This may be particularly preferred for treatment of a respiratory infection.

Suitable formulations, wherein the carrier is a liquid, for administration, as for example, a nasal spray or as nasal drops, include aqueous or oily solutions of the active ingredient. Alternatively, the AONs as described herein may be provided as a dry powder and administered to the upper respiratory tract only as defined herein.

The selection of appropriate carriers depends upon the particular type of administration that is contemplated. For administration via the upper respiratory tract, e.g. the nasal mucosal surfaces, the compound can be formulated into a solution, e.g. water or isotonic saline, buffered or unbuffered, or as a suspension, for intranasal administration as drops or as a spray. Preferably, such solutions or suspensions are isotonic relative to nasal secretions and of about the same pH, ranging e.g. from about pH 4.0 to about pH 7.4 or, from pH 6.0 to pH 7.0. Buffers should be physiologically compatible and include, simply by way of example, phosphate buffers. For example, a representative nasal decongestant is described as being buffered to a pH of about 6.2 (Remington's, Id. at page 1445). Of course, the ordinary artisan can readily determine a suitable saline content and pH for an innocuous aqueous carrier for nasal and/or upper respiratory administration.

Other ingredients, such as art known preservatives, colorants, lubricating or viscous mineral or vegetable oils, perfumes, natural or synthetic plant extracts such as aromatic oils, and humectants and viscosity enhancers such as, e.g. glycerol, can also be included to provide additional viscosity, moisture retention and a pleasant texture and odour for the formulation. For nasal administration of solutions or suspensions according to the methods of the invention, various devices are available in the art for the generation of drops, droplets and sprays. For example, an AON as described herein can be administered into the nasal passages by means of a simple dropper (or pipet) that includes a glass, plastic or metal dispensing tube from which the contents are expelled drop by drop by means of air pressure provided by a manually powered pump, e.g. a flexible rubber bulb, attached to one end.

A pharmaceutical composition may be formulated as inhaled formulations, including sprays, mists, or aerosols. This may be particularly preferred for treatment of a condition of the airway or lung involving smoke exposure as described herein. The inhaled formulation may be for application to the upper (including the nasal cavity, pharynx and larynx) and lower respiratory tract (including trachea, bronchi and lungs). For inhalation formulations, the composition or combination provided herein may be delivered via any inhalation methods known to a person skilled in the art. Such inhalation methods and devices include, but are not limited to, metered dose inhalers with propellants such as HFA or propellants that are physiologically and environmentally acceptable. Other suitable devices are breath operated inhalers, multidose dry powder inhalers and aerosol nebulizers. Aerosol formulations for use in the subject method typically include propellants, surfactants and co-solvents and may be filled into conventional aerosol containers that are closed by a suitable metering valve. Different devices and excipients can be used depending on whether the application is to the upper (including the nasal cavity, pharynx and larynx) or lower respiratory tract (including trachea, bronchi and lungs) and can be determined by those skilled in the art. Further, processes for micronisation and nanoparticle formation for the preparation of AONs described herein for use in an inhaler, such as a dry powder inhaler, are also known by those skilled in the art.

Inhalant compositions may comprise liquid or powdered compositions containing the active ingredient that are suitable for nebulization and intrabronchial use, or aerosol compositions administered via an aerosol unit dispensing metered doses. Suitable liquid compositions comprise the active ingredient in an aqueous, pharmaceutically acceptable inhalant solvent such as isotonic saline or bacteriostatic water. The solutions are administered by means of a pump or squeeze-actuated nebulized spray dispenser, or by any other conventional means for causing or enabling the requisite dosage amount of the liquid composition to be inhaled into the patient's lungs. Suitable formulations, wherein the carrier is a liquid, for administration, as for example, a nasal spray or as nasal drops, include aqueous or oily solutions of the active ingredient. Examples of inhalation drug delivery devices are described in Ibrahim et al. Medical Devices: Evidence and Research 2015:8 131-139, are contemplated for use in the present invention.

The delivery of a therapeutically useful amount of AONs may be achieved by methods previously published. For example, intracellular delivery of the AON may be via a composition comprising an admixture of the AON and an effective amount of a block copolymer. An example of this method is described in US patent application US20040248833. Other methods of delivery of AONs to the nucleus are described in Mann C J et al. (2001) Proc, Natl. Acad. Science, 98 (1) 42-47, and in Gebski et al. (2003) Human Molecular Genetics, 12 (15): 1801-1811. A method for introducing a nucleic acid molecule into a cell by way of an expression vector either as naked DNA or complexed to lipid carriers, is described in U.S. Pat. No. 6,806,084.

The AONs for use in the methods or uses of the present invention is to be administered in an effective amount. The phrase ‘therapeutically effective amount’ or ‘effective amount’ generally refers to an amount of an AON, a pharmaceutically acceptable salt, polymorph or prodrug thereof as described herein that (i) treats the particular disease, condition, or disorder, (ii) attenuates, ameliorates, or eliminates one or more symptoms of the particular disease, condition, or disorder, or (iii) delays the onset of one or more symptoms of the particular disease, condition, or disorder described herein. Undesirable effects, e.g. side effects, are sometimes manifested along with the desired therapeutic effect; hence, a practitioner balances the potential benefits against the potential risks in determining what is an appropriate “effective amount”.

The exact amount required will vary from subject to subject, depending on the species, age and general condition of the subject, mode of administration and the like. Thus, it may not be possible to specify an exact “effective amount”. However, an appropriate “effective amount” in any individual case may be determined by one of ordinary skill in the art using only routine experimentation. In one aspect, the dose administered to a subject is any dose that reduces viral load.

The phrase “pharmaceutically acceptable” refers to molecular entities and compositions that are physiologically tolerable and do not typically produce an allergic or similarly untoward reaction, such as gastric upset and the like, when administered to a patient. The term “carrier” refers to a diluent, adjuvant, excipient, or vehicle with which the compound is administered. Such pharmaceutical carriers can be sterile liquids, such as water and oils, including those of petroleum, animal, vegetable or synthetic origin, such as peanut oil, soybean oil, mineral oil, sesame oil and the like. Water or saline solutions and aqueous dextrose and glycerol solutions are preferably employed as carriers, particularly for injectable solutions. Suitable pharmaceutical carriers are described in Martin, Remington's Pharmaceutical Sciences, 18th Ed., Mack Publishing Co., Easton, PA, (1990).

Treatment may be monitored, e.g. by general indicators of disease known in the art. The efficacy of an in vivo administered AONs for use in the methods of the present invention may be determined from biological samples (tissue, blood, urine etc.) taken from a subject prior to, during and subsequent to administration of the AON. Assays of such samples include (1) monitoring the presence or absence of heteroduplex formation with target and non-target sequences, using procedures known to those skilled in the art, e.g. an electrophoretic gel mobility assay; (2) monitoring the amount of a mutant mRNA in relation to a reference normal mRNA or protein as determined by standard techniques such as RT-PCR, Northern blotting, ELISA or Western blotting.

The methods of the invention extends also to a combination of two or more AONs capable of binding to a selected target to induce exon exclusion in a RAGE gene transcript. The combination may be a cocktail of two or more AONs, a construct comprising two or more or two or more AONs joined together for use in an AON-based therapy. The combination of AONs is preferably a combination of SEQ ID NO: 11 and 10, or SEQ ID NO: 11 and 13.

Kits are also provided for use in the methods of the invention, in particular to treat, prevent or ameliorate a disease or condition associated with RAGE expression in a patient, which kit comprises at least an AON as described herein together with instructions for its use, wherein the instructions for use describe a method of the invention.

The contents of the kit can be lyophilized and the kit can additionally contain a suitable solvent for reconstitution of the lyophilized components. Individual components of the kit would be packaged in separate containers and, associated with such containers, can be a notice in the form prescribed by a governmental agency regulating the manufacture, use or sale of pharmaceuticals or biological products, which notice reflects approval by the agency of manufacture, use or sale for human administration.

As used herein the term “derived” and “derived from” shall be taken to indicate that a specific integer may be obtained from a particular source albeit not necessarily directly from that source.

As used herein, the singular forms “a,” “an” and “the” include plural references unless the context clearly dictates otherwise.

Other than in the operating example, or where otherwise indicated, all numbers expressing quantities of ingredients, reaction conditions, and so forth used in the specification and claims are to be understood as being modified in all instances by the term “about”. Accordingly, unless indicated to the contrary, the numerical parameters set forth in the specification and claims are approximations that may vary depending upon the desired properties sought to be obtained by the methods of the present invention. Hence “about 80%” means “about 80%” and also “80%”. At the very least, each numerical parameter should be construed in light of the number of significant digits and ordinary rounding approaches.

Notwithstanding that the numerical ranges and parameters setting forth the broad scope of the methods of the present invention are approximations, the numerical values set forth in the specific examples are reported as precisely as possible. Any numerical value; however, inherently contains certain errors necessarily resulting from the standard deviation found in their respective testing measurements

Other definitions for selected terms used herein may be found within the detailed description of the invention and apply throughout. Unless otherwise defined, all other scientific and technical terms used herein have the same meaning as commonly understood to one of ordinary skill in the art to which the invention belongs.

It will be understood that the invention disclosed and defined in this specification extends to all alternative combinations of two or more of the individual features mentioned or evident from the text or drawings. All of these different combinations constitute various alternative aspects of the invention.

It will be understood that the invention disclosed and defined in this specification extends to all alternative combinations of two or more of the individual features mentioned or evident from the text or drawings. All of these different combinations constitute various alternative aspects of the invention.

### EXAMPLES

### Example 1—Cigarette Smoke-Induced Inflammation Mouse Model Study Design

### Objective

To determine the anti-inflammatory activity of a single intratracheal dose of a fully-modified antisense oligonucleotide in mice exposed to cigarette smoke for four days.

### Protocol Outline

Three solutions labelled saline, ASO 6713 (3 mg/kg) and ASO 6713 (10 mg/kg) (SEQ ID NO: 33) were supplied and upon receipt materials were immediately stored at −20° C. in a temperature monitored freezer until required. Thirty minutes prior to use, solutions were thawed used at RT.

Mice were acclimatised for 1 week prior to experimental commencement. Following acclimatisation, mice were anaesthetised using a mixture of ketamine/xylazine (90 mg/10 mg/kg body weight via ip injection) and solutions administered when there was no reflex response (limb withdrawal to the hind paw). The tracheal opening was directly visualised using an otoscope/speculum device placed carefully into the mouth. Fifty microlitres of the allocated sterile solution was administered directly into the trachea through an aerosoliser microsprayer (Penn-Century IA-1C) connected to a high-pressure syringe (Penn-Century FMJ-250). Between each solution, the microsprayer and syringe was flushed. Mice were administered either ASO 6713 (3 mg/kg) or ASO 6713 (10 mg/kg) for groups as indicated in Table 2.

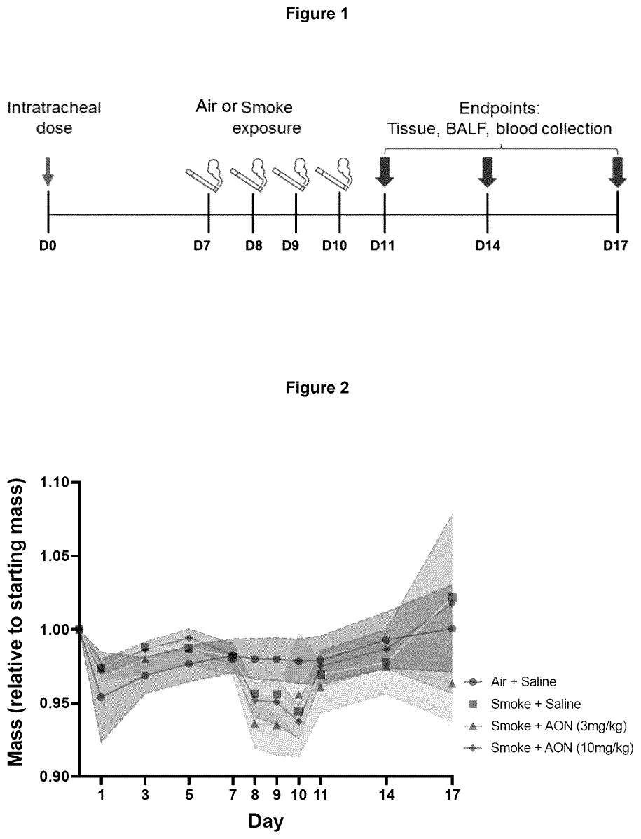

Following 1-week post-intratracheal administration, mice were exposed to cigarette smoke (CSE) for 4 days utilising a whole-body free roaming exposure chamber method. Specifically, mice were placed in an 18 L Perspex chamber in a standard fume hood cabinet and exposed to either air or Winfield Red Cigarettes (total particulate matter of ~419 mg m-3, ~16 mg of tar, ~1.2 mg of nicotine, and ~15 mg of CO; Philip Morris). CSE mice were exposed to 9 full cigarettes/day. Mice were exposed either air or CSE three times a day, with a 2-h break between smoke sessions. Following the exposure period, mice were humanely killed to be assessed for the following endpoints as outlined in FIG. 1.

### Animals

Specific pathogen-free male C57BI/6 mice (sourced at ~8 weeks old) were obtained from the Animal Resource Centre Pty. Ltd. (Perth, Australia). Mice were randomly assigned into 2 batches of 4 groups per endpoint (4 mice/group) and assessed at 3 endpoints (Table 2). The animals were housed in sterile passive micro-isolators at a constant 20° C. temperature on a 12-h day/night cycle and fed irradiated Barastoc mouse feed with irradiated tap water allowed ad libitum. Mouse weights were monitored throughout the experiment.

TABLE 2
Mouse treatment groups

| | Endpoint | C57BI/6 | No. | |
|---|---|---|---|---|
| | post IT | mice | mice/ | |
| Group | (D) | (~age) | group | Treatment |
| 1 | 11 | 20 | 4 | Untreated + Air |
| | | 20 | 4 | Smoke + Saline |
| | | 20 | 4 | Smoke + ASO 6713 (3 mg/kg) |
| | | 20 | 4 | Smoke + ASO 6713 |
| | | | | (10 mg/kg) |
| | 14 | 20 | 4 | Untreated + Air |
| | | 20 | 4 | Smoke + Saline |
| | | 20 | 4 | Smoke + ASo 6713 (3 mg/kg) |
| | | 20 | 4 | Smoke + ASO 6713 |
| | | | | (10 mg/kg) |
| | 17 | 20 | 4 | Untreated + Air |
| | | 20 | 4 | Smoke + Saline |
| | | 20 | 4 | Smoke + ASO 6713 (3 mg/kg) |
| | | 20 | 4 | Smoke + ASO 6713 |
| | | | | (10 mg/kg) |
| 2 | 11 | 20 | 4 | Untreated + Air |
| | | 20 | 4 | Smoke + Saline |
| | | 20 | 4 | Smoke + ASO 6713 (3 mg/kg) |
| | | 20 | 4 | Smoke + ASO 6713 |
| | | | | (10 mg/kg) |
| | 14 | 20 | 4 | Untreated + Air |
| | | 20 | 4 | Smoke + Saline |
| | | 20 | 4 | Smoke + ASO 6713 (3 mg/kg) |
| | | 20 | 4 | Smoke + ASO 6713 |
| | | | | (10 mg/kg) |
| | 17 | 20 | 4 | Untreated + Air |
| | | 20 | 4 | Smoke + Saline |
| | | 20 | 4 | Smoke + ASO 6713 (3 mg/kg) |
| | | 20 | 4 | Smoke + ASO 6713 |
| | | | | (10 mg/kg) |

### Plasma Collection

Mice were exsanguinated by drawing blood from the inferior vena cava with a 19G syringe lined with lithium heparin into a Microvette lithium/heparin tube (Sarstedt, Aust.). Blood was maintained at RT for a minimum of 15 minutes and centrifuged at 2000 g for 5 minutes at 20° C. Plasma was transferred into 1.5 mL snap-lock Eppendorf tubes, snap frozen in liquid nitrogen and stored at −80° C.

### Bronchoalveolar Lavage and Differential Counts

Bronchoalveolar lavage (BAL) was performed via a surgical tracheotomy and lavaged in situ with 0.4 mL of chilled PBS, followed by further 0.3 mL aliquots of PBS until approximately 1 mL of BAL fluid (BALF) was collected. Cytocentrifuge spots were created by centrifuging the BALF at 400×g for 10 min. The cytospots were stained with Shandon Kwik-Diff Kit® and Merck's Hemacolor as previously described. Differential counts were performed, counting a total of 200 cells identifying macrophages+monocytes, neutrophils+eosinophils, and lymphocytes using standard morphological criteria.

### Tissue Collection

Lungs were perfused to remove circulating blood. A 20G needle was inserted into the right ventricle and 5-10 mL of chilled, sterile PBS was administered at a rate of 300 μl/second. The lungs were then excised as previously described. The left lobe was divided into upper, lower left (LL) and lower right (LR) portions and collected in sterile 1.5 mL snap-lock Eppendorf tubes. The superior right, inferior right and post-caval lobes were collected in a sterile 1.7 mL snap-lock Eppendorf tube. All lung tissue was immediately snap frozen in liquid nitrogen. Snap frozen tissue was transferred to −80° C. for storage.

Measuring Levels of RAGE_v1 mRNA Expression in Lung Tissue

To measure the effect of interventions on the alternative splicing of RAGE pre-mRNA, the expression of RAGE mRNA splice variants was determined using real-time RT-qPCR, performed using the TaqMan system based on real-time detection of accumulated fluorescence (ABI Prism 7700, Perkin-Elmer Inc, PE Biosystems, Foster City, CA, USA) as previously utilized (43). For in vivo experiments, frozen lung tissue from the left lower lobe was used. RNA extraction cDNA synthesized was performed using the Trizol. Gene expression was estimated by RT-qPCR, performed using either the Taqman or SYBR Green (Sigma) system on the basis of real-time detection of accumulation of fluorescence (Applied Biosystems Q3 and Q5). Gene Expression was normalized to 18S mRNA and reported as fold change compared to the level of expression in control RNA treated cells, which were given an arbitrary value of 1. To define the expression of RAGE mRNA splice variants retaining exon 9b a probe was designed to span exon 9b. To define the expression of RAGE mRNA splice variants retaining exon 10, a PCR primer/probes spanning exon 8-10 were designed. To define the expression of all RAGE mRNA, PCR probe/primers spanning exon 8 and 11 were designed. A reduction in the signal of exon 8-10 of the cytosolic tail relative to exon 8-11 indicates that less full-length RAGE transcript (signalling capable) is produced by the cell.

### Example 2—Analysis of Broncho-Alveolar Lavage Fluid (BALF) Cell Counts in a Smoke-Induced Inflammation Mouse Model

In this experiment, mice (8 per group) were administered saline, or ASO 6713 (3 mg/kg or 10 mg/kg) to the left lobe of the lung using an aerosolised microsprayer and exposed to cigarette smoke 1 week later as described above. Mice were humanely killed at day 11, 14 or 17 following saline or ASO 6713 treatment. Broncho-alveolar lavage fluid (BALF) was collected from the left lobe of the mice lungs for analysis.

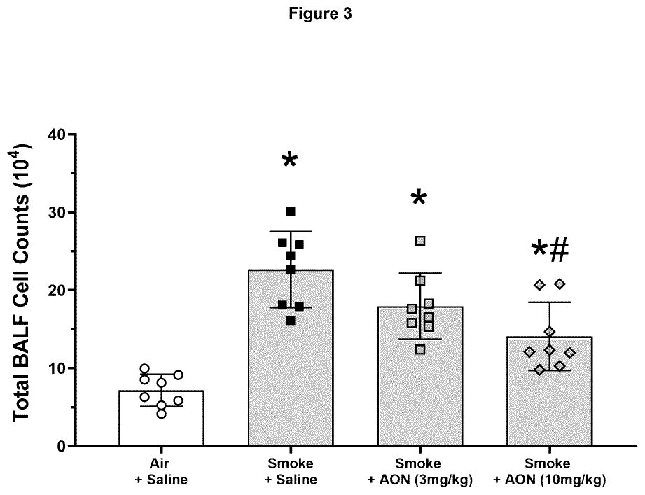

All mice exposed to smoke experienced a modest acute weight loss that was fully reversible when smoke exposure was discontinue, consistent with the anorexic effect of nicotine (FIG. 2).

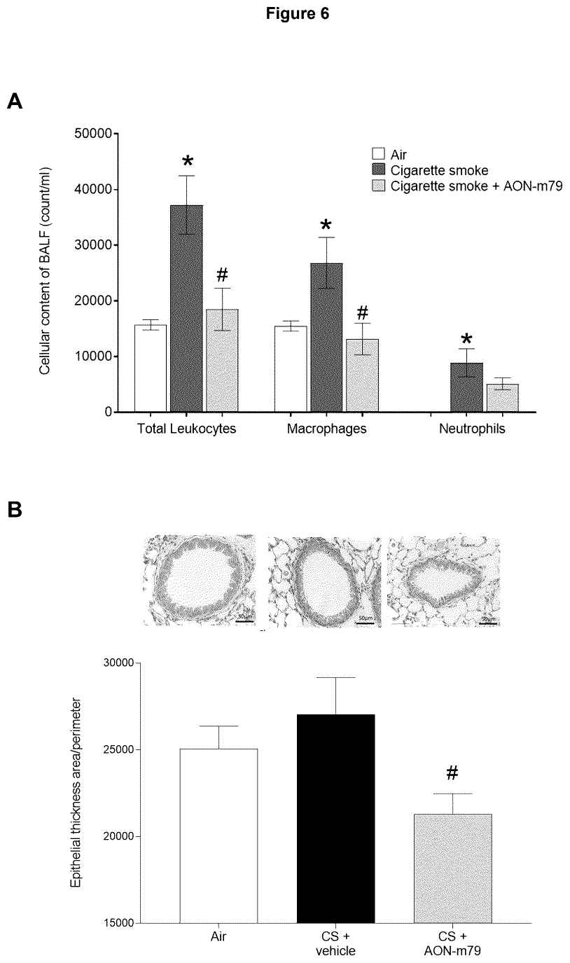

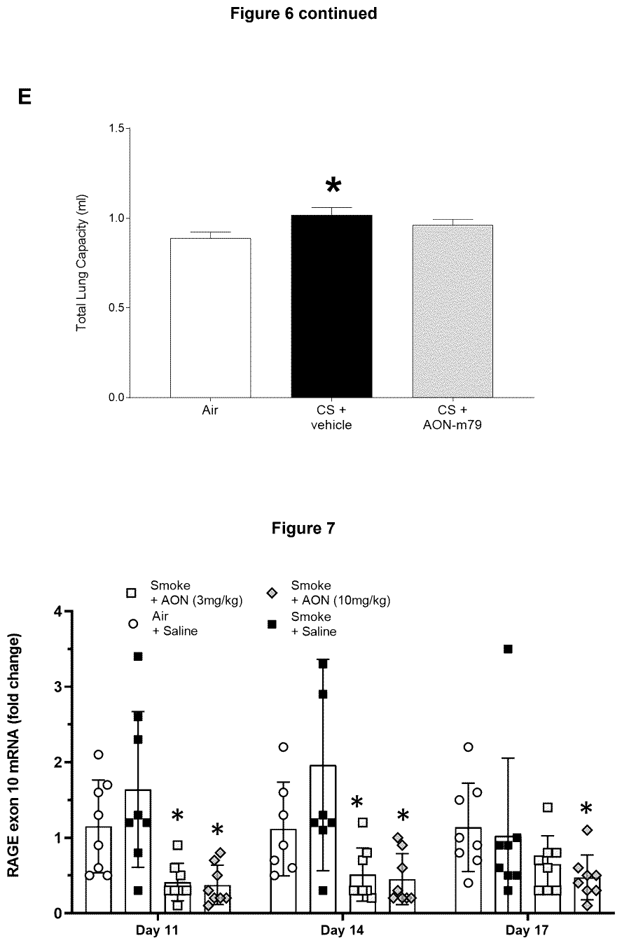

Low dose (3 mg/kg) and high dose (10 mg/kg) ASO 6713 treatment was shown to increase RAGE_v1 mRNA expression (encoding esRAGE protein) in mice exposed to cigarette smoke as measured as days 11, 14 and 17 post-ASO 6713 treatment (FIG. 5). Consistent with the splice switching activity of ASO 6713, RAGE mRNA levels containing exon 10 were found to decrease at days 11, 13 and 17 with ASO 6713 treatment (FIG. 7).

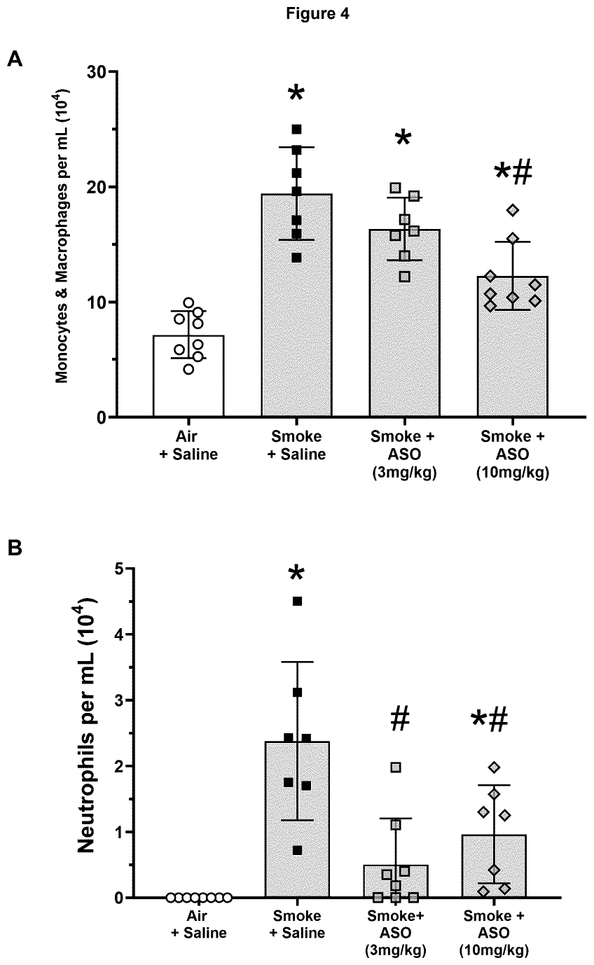

Total cell counts in BALF in mice lungs at day 11 increased following smoke exposure compared to the control group, however, an initial high dose of ASO 6713 (10 mg/kg) prior to smoke exposure was able to significantly attenuate the increase in BALF cell counts (FIG. 3). BALF cell counts were observed to slowly decrease in the smoke-exposed untreated mice group from day 11 to 14 and was not significantly different to the non-smoke exposed group at day 14 (data not shown).

### Example 3—Analysis of Immune Cell Subtypes in BALF Samples Collected from Smoke Exposed Mice

The inventors then performed a differential cell count for immune cells within the BALF to determine whether the increased BALF cell counts were due to an increase in the number of inflammatory immune cells present in the lung.

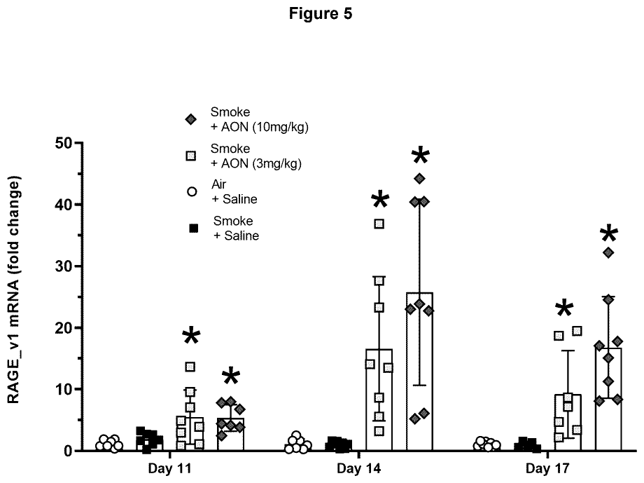

As expected, the smoke exposed group displayed an increased infiltration of interstitial (non-alveolar) macrophages and neutrophils compared to the non-smoke exposed group at day 11 (FIG. 4). Consistent with the findings above, the high dose ASO 6713 treatment was able to suppress the infiltration of these inflammatory cells. Low dose ASO 6713 treatment was also shown to significantly suppress the infiltration of neutrophils into the left lung.

### Example 4—Cigarette Smoke-Induced Experimental COPD Study Design

### Animals

All animal care and experimental procedures were conducted in accordance with the local Guidelines and were approved by the animal ethics committee of the Sydney Local Health District, NSW, Australia. Female wild-type C57BL/6 mice were obtained from Australian BioResources Ltd (Mossvale, NSW, Australia) and group housed (5 mice per cage) under specific pathogen-free conditions at Centenary Institute, NSW, Australia. The light/dark cycle was 12/12 h; temperature range 22°-26° C. and humidity range 30-70% relative humidity. Mice were randomly numbered and then separated into the experimental groups. All tissues and samples were processed using this number as the identifier. Assays were performed without knowing which number belonged to which group. Once the assay was completed, this number was used to identify the experimental group providing the sample.

### Protocol Outline

Female C57BL/6 mice (6-8 weeks old) were exposed either to cigarette smoke from 12 research-grade cigarettes (3R4F, University of Kentucky, Lexington, KY, USA; each cigarette was smoked over 5 minutes) or to normal air for 75 minutes, twice per day for 8 weeks. Cigarette smoke was delivered to mice using an in-house custom-designed and purpose-built nose-only, directed-flow inhalation and smoke-exposure system (CH Technologies, Westwood, NJ, USA).

To test the therapeutic potential of an AON that is complementary to a pre-mRNA target region that has a 5′-most nucleotide at position 88 or 114 of exon 10 or that is between nucleotide positions 88 to 114 of exon 10, AON-m79 was chosen. Mice were treated with AON-m79 (3 mg/kg, once weekly) or vehicle control via intranasal instillation, with the first treatment delivered on the day of first exposure to cigarette smoke and continued weekly for the duration of the study.

### Bronchoalveolar Lavage and Differential Counts

Airway inflammation was assessed by enumerating total leukocyte cell numbers as well as specific inflammatory cell types in BAL fluid. BAL fluid was obtained by lavaging lungs with two 400 μL aliquots of PBS at room temperature. Total cell numbers were assessed using Trypan Blue exclusion. Cytospins were performed on the remaining BAL fluid cells, and differential cell counts were obtained based on morphology.

### Lung Tissue Staining

Sections (4 μm thick) of paraffin-embedded, formalin-fixed, inflated lung tissue were mounted on microscope slides and stained with haematoxylin and eosin. Epithelial thickness was assessed by measuring the difference in area of the epithelial cell basement membrane and lumen. For this purpose, photographs were taken of the stained lung sections and visualised using Image J software (version 1.47).

Small airway-associated fibrosis was assessed in formalin-fixed lung sections by measuring the deposition of Masson's Trichrome Blue-stained collagen around the small airways. Stained lung sections on slides were photographed, and the amount of collagen was calculated by blinded observers using Image J software (version 1.47) by assessing the area of deposition around small airways of <1 mm diameter using the formula (Wct/Pbm), where Wct=AoAi, inner collagen area=Ai, outer collagen area=Ao and basement membrane length=Pbm.

Sections (4 μm thick) of paraffin-embedded, formalin-fixed, inflated lung tissue were mounted on microscope slides and stained with haematoxylin and eosin. Emphysema-like alveolar enlargement was assessed using the mean linear intercept (MLI) technique, which is a standard method for assessing alveolar size and emphysema in mice. A standardized template containing horizontal lines was laid over micrographs of lung sections. The number of alveolar intercepts was counted, and the resulting average number of intercepts was determined.

### Total Lung Capacity Assessment

Total lung capacity (TLC) was assessed on the day of endpoint. Mice were anaesthetised with ketamine and xylazine hydrochloride in PBS then tracheostomised, cannulated and connected to the flexiVent FX2 (SCIREQ Scientific Respiratory Equipment Inc., Montreal, Canada) system and operated by flexiWare version 8 software. The mice were mechanically ventilated, and the lung volumes manoeuvre was performed, in which the lungs were degassed with 100% oxygen to allow inflation to start at zero volume. The lungs were then inflated with 3 full range pressure volume loop ramp-style inflations and deflations in order to calculate TLC.

### Statistical Analysis

Data shown are means±SEM (n=6-8). All datasets were determined to be normally distributed based on previous experiments and were also tested using the Kolmogorov-Smirnov test (α=0.05). Data were analysed using one-way ANOVA with Tukey's multiple comparison test using PRISM software (V6.0d, GraphPad, La Jolla, CA, USA), with P<0.05 considered statistically significant.

### Example 4—AON m79 Treatment Attenuates Features of Smoke-Induced Experimental Chronic Obstructive Pulmonary Disease (COPD)

To determine whether RAGE splice switching oligonucleotides could be used to prevent smoke induced lung damage, Female C57BL/6 mice were exposed to cigarette smoke from 12 3R4F reference cigarettes twice/day, 5 times/week on consecutive days for 8 weeks. This exposure is approximately equivalent to a pack-a-day human smoker and is known to induce pathophysiological features consistent with human COPD, including the induction of persistent airways inflammation dominated by and macrophages and neutrophils, which drive the pathology and development of disease features.

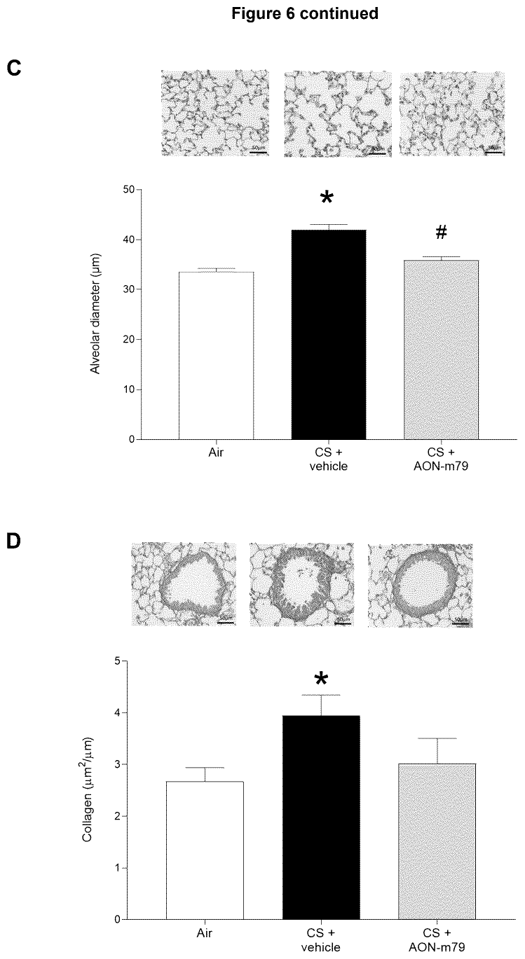

Intranasal treatment with AON-m79 (3 mg/kg/once-weekly) significantly prevented the increases in inflammatory cells in the bronchoalveolar (BAL) fluid associated with smoke exposure (FIG. 6A). Indeed, treatment completely inhibited the influx of macrophages that were at baseline levels in normal air-exposed controls. There was also a reduction in lymphocytes (cigarette smoke (CS); mean=425 cells/mL of BAL fluid vs CS+AON m79; mean=189.8 cells/mL, p=0.05) and eosinophils (CS; mean=839.57 cells/mL vs CS+AON-m79; mean=78.56 cells/mL, p=0.05) in BAL fluid following treatment with AON-m79. Smoke-induced lung injury in this 8-week model was associated with airway remodelling with increases in epithelial thickness (FIG. 6B), emphysema-like alveolar enlargement (FIG. 6C) and fibrosis around the small airways (FIG. 6D) characteristic pathological feature of early human COPD. In combination, these changes were associated with reduced lung function (FIG. 6E). Treatment with AON-m79 substantially prevented each of these airway remodelling, emphysematous and lung function changes that are hallmark chronic features of human COPD.

Together these results suggest that RAGE splice switching oligonucleotide treatment is able to protect the lung against an increase in inflammatory and hallmark features associated with the development of COPD.

### Example 5—Cigarette Smoke-Induced COPD Mouse Model Extended Exposure Study Design

### Protocol Outline

Two solutions labelled saline and ASO 6713 (3 mg/kg) were supplied and upon receipt materials were immediately stored at −20° C. in a temperature monitored freezer until required. Thirty minutes prior to use, solutions were thawed used at RT.

C57BI/6 mice at 8 weeks of age were randomly allocated into groups as shown in Table 2. Mice were acclimatised for 1 week prior to experimental commencement. Following acclimatisation, all mice were exposed to cigarette smoke (CSE) for 4 or 8 months utilising a whole-body free roaming exposure chamber method. Specifically, mice were placed in an 18 L Perspex chamber in a standard fume hood cabinet and exposed to either air or Winfield Red Cigarettes (total particulate matter of ~419 mg m-3, ~16 mg of tar, ~1.2 mg of nicotine, and ~15 mg of CO; Philip Morris). CSE mice were exposed to 9 full cigarettes/day. Mice were exposed CSE three times a day, with a 2-h break between smoke sessions.

For groups 1, 2 and 3 a single intratracheal dose of the allocated is provided one week prior to commenced of CSE (or control air exposure). Briefly, mice were anaesthetised using a mixture of ketamine/xylazine (90 mg/10 mg/kg body weight via ip injection) and solutions administered when there was no reflex response (limb withdrawal to the hind paw). The tracheal opening was directly visualised using an otoscope/speculum device placed carefully into the mouth. Fifty microlitres of the allocated sterile solution or ASO 6713 solution was administered directly into the trachea through an aerosoliser microsprayer (Penn-Century IA-1C) connected to a high-pressure syringe (Penn-Century FMJ-250). Between each solution, the microsprayer and syringe was flushed.

Weekly intranasal dosing commenced at the same time as CSE or control air exposure and was delivered in fifty microliters volume to the nostrils of anaesthetised mice by pipette.

Following 4 months CSE, smoke exposure was ceased for groups 5 and 6, and 4 mice from each group were humanely killed and assessed by histology to confirm the occurrence of pre-COPD. At the same time, the remaining mice for groups 4, 5 and 6, commenced weekly intranasal doses of saline or ASO 6713 for 4 months using the same intranasal administration protocol described above.

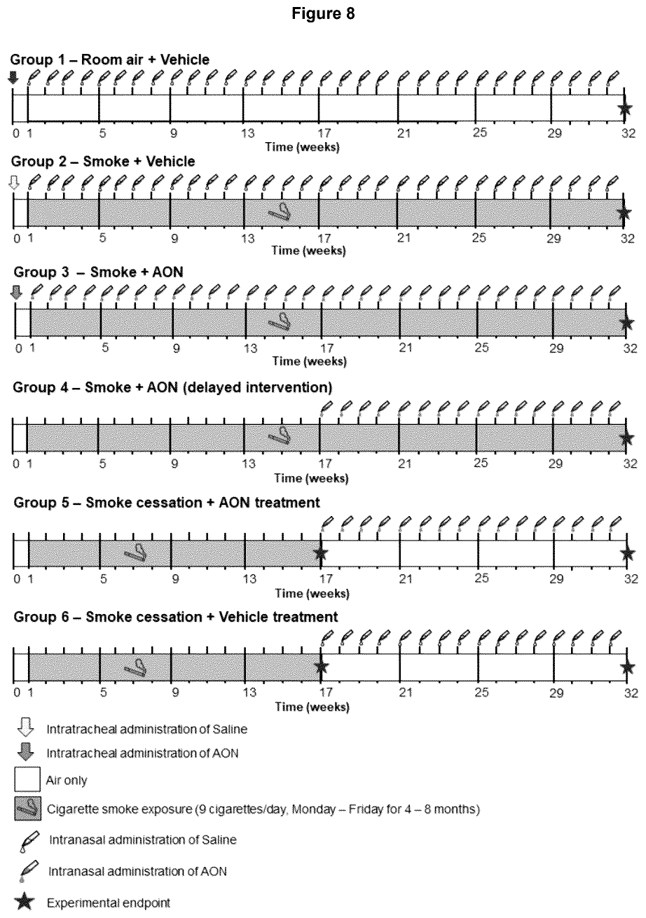

At 8 months CSE, all groups of mice were humanely killed and assessed. A schematic of the experimental protocol is shown in FIG. 8.

TABLE 3
Experimental outline

| Group | Mice/group | Treatment |
|---|---|---|
| Group 1 | 12 | 8 months normal air exposure + saline single |
| Negative control (Air) | | intratracheal, (50 μL) followed by weekly intranasal |
| | | dosing |
| Group 2 | 12 | 8 months CSE + saline single intratracheal, (50 μL) |
| Positive control (CSE) | | followed by weekly intranasal dosing |
| Group 3 | 12 | 8 months CSE + ASO6713 single intratracheal, (3 |
| CSE + ASO | | mg/kg)) followed by weekly intranasal dosing |
| Group 4 | 12 | 8 months CSE + 4 months ASO6713 weekly intranasal |
| CSE + ASO delayed | | (3 mg/kg) dosing following smoke cessation |
| Group 5 | 16 | 4 months CSE + 4 months ASO6713 weekly intranasal |
| Smoking cessation + ASO | | (3 mg/kg) dosing following smoke cessation |
| Group 6 | 16 | 4 months CSE + 4 months weekly intranasal saline |
| Smoking cessation + saline | | (50 μL) dosing following smoke cessation |

### Animals

Specific pathogen-free female C57BL/6 mice (sourced at ~8 weeks old) were obtained from the Animal Resource Centre Pty. Ltd. (Perth, Australia). The animals were housed in sterile passive micro-isolators at a constant 20° C. temperature on a 12-h day/night cycle and fed irradiated Barastoc mouse feed with irradiated tap water allowed ad libitum. Mouse weights were monitored and recorded on days 0, 1, 3 and then daily during smoke exposure and following smoking cessation.

### Mean Linear Intercept

At experimental endpoint the mice were humanely killed. Lungs were inflated and fixed with 10% neutral buffered formalin under a constant pressure (20 cm H₂O) and paraffin-embedded. Sections (4 μm thick) of paraffin-embedded, formalin-fixed, inflated lung tissue were mounted on microscope slides and stained with haematoxylin and eosin. Emphysema-like alveolar enlargement was assessed using the mean linear intercept (MLI) technique, which is a standard method for assessing alveolar size and emphysema in mice. A standardized template containing horizontal lines was laid over micrographs of lung sections. The number of alveolar intercepts was counted, and the resulting average number of intercepts was determined.

### Statistical Analysis

Data shown are means±SEM. All datasets were determined to be normally distributed based on previous experiments and were also tested using the Kolmogorov-Smirnov test (α=0.05). Data were analysed using one-way ANOVA with Tukey's multiple comparison test using PRISM software (V6.0d, GraphPad, La Jolla, CA, USA), with P<0.05 considered statistically significant.

### Example 6—ASO 6713 Treatment Attenuates Emphysema-Like Alveolar Enlargement with Continued Smoke Exposure

To determine whether RAGE splice switching oligonucleotides could be used to prevent smoke induced lung damage, male C57BL/6 mice were exposed to the equivalent of 9 cigarettes/day for up to 8 months according to the experimental protocol outlined in Example 5 above. Weakly intranasal administration of ASO 6713 (3 mg/kg) had no negative effects on body mass monitoring, nor body condition score over the 8-month period (data not shown).

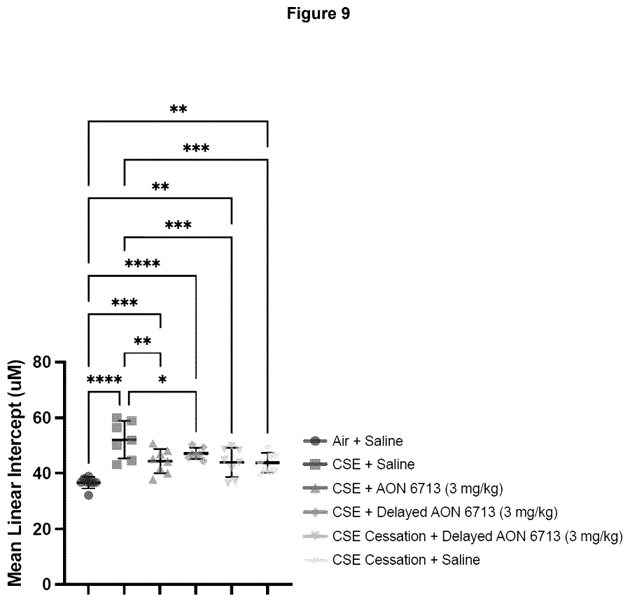

An initial priming inhaled dose prior to CSE followed by weekly intranasal dosing of ASO 6713 (3 mg/kg) was found to significantly prevent the emphysema-like alveolar enlargement associated with smoke exposure (FIG. 9).

Emphysema-like alveolar enlargement was also found to be significantly reduced in mice where ASO 6713 treatment has been delayed until after an initial 4 months of smoke exposure (FIG. 9). Surprisingly, even though this treatment group were continually exposed to smoke for another 4 months with concurrent ASO 6713 treatment, the mice exhibited similar alveolar size to the treatment group that ceased smoke exposure after 4 months (FIG. 9; cf. CSE+Delayed AON 6713 v CSE cessation+saline).

Together these results suggest that RAGE splice switching oligonucleotide treatment is able to protect and treat smoke induced emphysema-like structural changes to the lung, even in individuals that are established smokers or are unable to cease smoking.

1. A method of treating or preventing obstructive pulmonary disease, associated with or caused by exposure to smoke, in a subject in need thereof, the method comprising administering to the subject an antisense oligonucleotide (AON) that promotes the production of endogenous soluble RAGE and/or reduces the production of membrane bound RAGE (mRAGE), treating or preventing obstructive pulmonary disease, associated with or caused by exposure to smoke in the subject.

2. A method of alleviating or ameliorating a symptom of obstructive pulmonary disease, associated with or caused by exposure to smoke, in a subject in need thereof, the method comprising administering to the subject in need thereof an antisense oligonucleotide (AON) that promotes the production of endogenous soluble RAGE and/or reduces the production of membrane bound RAGE (mRAGE), thereby alleviating or ameliorating a symptom of obstructive pulmonary disease, associated with or caused by exposure to smoke in the subject.

3. Use of an antisense oligonucleotide (AON) that promotes the production of endogenous soluble RAGE and/or reduces the production of membrane bound RAGE (mRAGE) in the manufacture of a medicament for the treatment or prevention of obstructive pulmonary disease, associated with or caused by exposure to smoke, in a subject in need thereof.

4. A method or use according to any one of the preceding claims, wherein the AON that promotes the production of endogenous soluble RAGE and/or reduces the production of membrane bound RAGE (mRAGE) is administered directly to the airway and/or lungs.

5. A method or use according to any one of the preceding claims, wherein the administration of the AON promotes the production of endogenous soluble RAGE and/or reduces the production of membrane bound RAGE (mRAGE) in the total respiratory tract, lower respiratory tract or upper respiratory tract.

6. A method or use according to any one of the preceding claims, wherein the method promotes the production of endogenous soluble RAGE and/or reduces the production of membrane bound RAGE (mRAGE) in the lower respiratory tract, preferably the lung.

7. A method or use according to any one of the preceding claims, wherein the antisense oligonucleotide (AON) that promotes the production of endogenous soluble RAGE and/or reduces the production of membrane bound RAGE (mRAGE) is administered via inhalation.

8. A method or use according to any one of the preceding claims, wherein the antisense oligonucleotide (AON) that promotes the production of endogenous soluble RAGE and/or reduces the production of membrane bound RAGE (mRAGE) is administered via intranasal administration.

9. A method or use according to any one of the preceding claims, wherein the obstructive pulmonary disease is chronic obstructive pulmonary disease (COPD), emphysema, combined pulmonary fibrosis and emphysema (CPFE), bronchitis, respiratory bronchiolitis/respiratory bronchiolitis-interstitial lung disease, bronchiectasis, asthma, lung, tracheal cancer or a smoking-related interstitial lung diseases (SR-ILDs), preferably the chronic obstructive smoking-related interstitial lung diseases (SR-ILDs) is pulmonary Langerhans cell histiocytosis, desquamative interstitial pneumonitis, or acute eosinophilic pneumonia.

10. The method of any one of claims 1 to 9, wherein the AON promotes the production of endogenous soluble RAGE by promoting the inclusion of exon 9b and/or the exclusion (e.g. skipping) of exon 10.

11. The method of any one of claims 1 to 19 wherein AON promotes splicing in the RAGE pre-mRNA resulting in the inclusion of exon 9b and/or skipping of exon 10.

12. The method of claim 10 or 11, wherein administration of the AON results in an increase of the level of RAGE_v1, preferably an increase in the level of RAGE_v1 mRNA in one or more tissues of the respiratory tract.

13. The method of any one of claims 1 to 12, wherein the AON is administered to the total respiratory tract, the upper respiratory tract or the lower respiratory tract.

14. The method of any one of claims 1 to 13, wherein the AON is administered as an aerosol.

15. The method of any one of claims 1 to 14, wherein the AON is administered in a single dose.

16. The method of any one of claims 1 to 15, wherein the AON is an AON of 10 to 50 nucleotides comprising a targeting sequence complementary to a region near or within an intron of the RAGE pre-mRNA, optionally, the AON is 10 to 50 nucleotides comprising a targeting sequence complementary or adjacent to a splice site of the RAGE pre-mRNA.

17. The method of any one of claims 1 to 16, wherein, the AON is 10 to 50 nucleotides comprising a targeting sequence complementary to RAGE pre-mRNA which modulates secondary structure of said mRNA to influence splice site selection.

18. The method of any one of claims 1 to 17, wherein the AON is an isolated or purified AON for inducing exclusion (also known as skipping) of one or more exonic sequences in the RAGE gene transcript or part thereof.

19. The method of any one of claims 1 to 18, wherein the AON is an isolated or purified AON for inducing retention of intronic sequences in the RAGE gene transcript or part thereof.

20. The method of any one of claims 1 to 19, wherein the AON comprises at least one modified nucleotide.

21. The method of any one of claims 1 to 20, wherein the AON is chemically-modified to prevent degradation of the pre-mRNA-AON complex, preferably wherein the chemical modification is selected from the group consisting of: phosphorodiamidate morpholino oligomers (PMO), 2′ O-methyl phosphorothioate oligonucleotides (20Me), and 2′-O-methoxyethyl phosphorothioate oligonucleotides (2′-MOE), locked nucleic acid (LNA) modified AONs, thermostable twisted intercalating nucleic acid (TINA) and peptide nucleic acids (PNAs).

22. The method of any one of claims 1 to 21, wherein the AON is conjugated to moieties to increase its delivery, preferably cell-penetrating peptides (CPPs), vivo-morpholinos (VMO) or peptide phosphorodiamidate morpholino oligomers (PPMO).

23. The method of any one of claims 1 to 22, wherein the AON comprises, consists essentially of or consists of a nucleotide sequence at least 80%, 81%, 82%, 83%, 84%, 85%, 86%, 87%, 88%, 89%, 90%, 91%, 92%, 93%, 94%, 95%, 96%, 97%, 98%, 99% or 100% complementary to a target region of exon 10 of RAGE pre-mRNA over the entirety of the antisense oligonucleotide.

24. The method of claim 23, wherein the 5′-most nucleotide of the target region is nucleotide position 88 or 114 of exon 10 or is between nucleotide positions 88 to 114 of exon 10.

25. The method of any one of claims 1 to 24, wherein the AON is 8 to 40 nucleotides in length, 15 to 25 nucleotides in length or 18 nucleotides in length.

26. The method of any one of claims 1 to 25, wherein the AON is selected from the group comprising the sequences set forth in any of Tables 1a-1d.

27. The method of any one of claims 1 to 26, wherein the AON comprises the nucleotide sequence as set forth in any one of SEQ ID NO: 1-31 and 33 or a nucleotide sequence at least 85%, 90% or 95% identical thereto.

28. The method of claim 27, wherein the AON comprises the nucleotide sequence as set forth in SEQ ID NO: 11, 18, 19, or 20, or a nucleotide sequence at least 85%, 90% or 95% identical thereto.

29. The method of any one of the preceding claims, wherein the smoke is aerosol or particulate matter from one or more of cigarettes, tobacco or marijuana, coal, wood, oil, gas or other biomass fires.

30. The method of any one of the preceding claims, wherein the smoke is cigarette smoke.

31. The method of any one of the preceding claims, wherein the cigarette smoke is “second-hand smoke”.

32. The method of any one of the preceding claims, wherein the smoke is from burnt organic matter, coal dust, smog, exhaust fumes and other industrial pollutants.

33. The method of any one of the preceding claims, wherein the subject has experienced an acute exposure to smoke.

34. The method of any one of claims 1 to 29, wherein the subject has been exposed to wood or forest fire smoke.

35. The method of any one of claims 1 to 29, wherein the obstructive pulmonary disease is smoke-induced COPD.

36. The method of any one of claims 1 to 35, wherein the subject is currently being exposed to smoke.

37. The method of claim 36, wherein the subject is currently a cigarette or tobacco smoker, or vaper.
